(19) United States
(12) Patent Application Publication
(10) Pub. No.: US 2025/0041283 A1
(43) Pub. Date: Feb. 6, 2025

(54) COCRYSTAL OF AN IDH1 INHIBITOR, PROCESS OF PREPARATION THEREOF, PHARMACEUTICAL COMPOSITIONS THEREOF, AND METHODS OF TREATMENT INVOLVING THE SAME

(71) Applicant: Servier Pharmaceuticals LLC, Boston, MA (US)

(72) Inventors: Nicolas LEBLANC, Lion-en-Beauce (FR); Patrick COINTEPAS, Saint Jean de la Ruelle (FR); Quentin VIEL, Canteleu (FR); Vincent ESCOLIVET, Yvetot (FR)

(21) Appl. No.: 18/791,857

(22) Filed: Aug. 1, 2024

(30) Foreign Application Priority Data
Aug. 1, 2023 (EP) 23306322.1

(51) Int. Cl. A61K 31/444; A61K 9/20; C07D 401/14

(52) U.S. Cl. CPC A61K 31/444; A61K 9/2009; A61K 9/2013; A61K 9/2054; C07D 401/14

(57) ABSTRACT

Provided is a cocrystal of a compound useful for treating cancer and a process for the preparation thereof, pharmaceutical compositions thereof and use for the treatment of cancer comprising administering the cocrystal described herein to a patient in need thereof.

### BACKGROUND

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2,4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production.

The human IDH1 gene encodes a protein of 414 amino acids. The nucleotide and amino acid sequences for human IDH1 can be found as GenBank entries NM_005896.2 and NP_005887.2 respectively. The nucleotide and amino acid sequences for IDH1 are also described in, e.g., Nekrutenko et al., Mol. Biol. Evol. 15:1674-1684(1998); Geisbrecht et al., J. Biol. Chem. 274:30527-30533(1999); Wiemann et al., Genome Res. 11:422-435(2001); The MGC Project Team, Genome Res. 14:2121-2127(2004); Lubec et al., Submitted (DEC-2008) to UniProtKB; Kullmann et al., Submitted (JUN-1996) to the EMBL/GenBank/DDBJ databases; and Sjoeblom et al., Science 314:268-274(2006).

Non-mutant, e.g., wild type, IDH1 catalyzes the oxidative decarboxylation of isocitrate to α-ketoglutarate, thereby reducing NAD⁺ (NADP⁺) to NADH (NADPH), e.g., in the forward reaction:

Isocitrate+NAD⁺(NADP⁺)→α-KG+CO₂+NADH(NADPH)+H⁺.

It has been discovered that mutations of IDH1 present in certain cancer cells result in a new ability of the enzyme to catalyze the NADPH-dependent reduction of α-ketoglutarate to R(−)-2-hydroxyglutarate (2HG). The production of 2HG is believed to contribute to the formation and progression of cancer (Dang, L et al., Nature 2009, 462:739-44).

The inhibition of mutant IDH1 and its neoactivity is therefore a potential therapeutic treatment for cancer. Accordingly, there is an ongoing need for inhibitors of IDH1 mutants having alpha hydroxyl neoactivity.

PCT publication WO2013/107291 discloses compounds that inhibit IDH1 mutants (e.g., IDH1R132H or IDH1R132C), in particular (S)—N—((S)-1-(2-chlorophenyl)-2-((3,3-difluorocyclobutyl)amino)-2-oxoethyl)-1-(4-cyanopyridin-2-yl)-N-(5-fluoropyridin-3-yl)-5-oxopyrrolidine-2-carboxamide. This application additionally discloses methods for the preparation of mutant IDH1 inhibitors, pharmaceutical compositions containing these compounds, and methods for the therapy of diseases, disorders or conditions (e.g., cancer) associated with overexpression and/or amplification of mutant IDH1.

PCT publication WO2015/138839 further discloses crystalline Forms 1 and 2 of (S)—N—((S)-1-(2-chlorophenyl)-2-((3,3-difluorocyclobutyl)amino)-2-oxoethyl)-1-(4-cyanopyridin-2-yl)-N-(5-fluoropyridin-3-yl)-5-oxopyrrolidine-2-carboxamide, solid dispersion prepared from these crystalline forms, especially Form 1, tablets prepared from such solid dispersion, as well as methods of treatment of advanced hematologic malignancies using such solid dispersions.

PCT publication WO2020/010058 discloses various solid state forms of (S)—N—((S)-1-(2-chlorophenyl)-2-((3,3-difluorocyclobutyl)amino)-2-oxoethyl)-1-(4-cyanopyridin-2-yl)-N-(5-fluoropyridin-3-yl)-5-oxopyrrolidine-2-carboxamide.

PCT publication WO2021/026436 discloses methods for the preparation of (S)—N—((S)-1-(2-chlorophenyl)-2-((3,3-difluorocyclobutyl)amino)-2-oxoethyl)-1-(4-cyanopyridin-2-yl)-N-(5-fluoropyridin-3-yl)-5-oxopyrrolidine-2-carboxamide, as well as crystalline ethanol solvate of this compound

There is a need for pharmaceutical compositions containing (S)—N—((S)-1-(2-chlorophenyl)-2-((3,3-difluorocyclobutyl)amino)-2-oxoethyl)-1-(4-cyanopyridin-2-yl)-N-(5-fluoropyridin-3-yl)-5-oxopyrrolidine-2-carboxamide that would have properties suitable for large-scale manufacturing and formulation, as well as utility in treating, by oral route, advanced solid tumors, such as glioma, intrahepatic cholangiocarcinomas (IHCC), chondrosarcoma, prostate cancer, colon cancer, melanoma, or non-small cell lung cancer (NSCLC), each characterized by the presence of a mutant allele of IDH1.

There is also a need for pharmaceutical compositions containing (S)—N—((S)-1-(2-chlorophenyl)-2-((3,3-difluorocyclobutyl)amino)-2-oxoethyl)-1-(4-cyanopyridin-2-yl)-N-(5-fluoropyridin-3-yl)-5-oxopyrrolidine-2-carboxamide that would have properties suitable for large-scale manufacturing and formulation, as well as utility in treating, by oral route, advanced hematologic malignancies, such as acute myelogenous leukemia (AML), myelodysplastic syndrome (MDS), myeloproliferative neoplasms (MPN), chronic myelomonocytic leukemia (CMML), B-acute lymphoblastic leukemias (B-ALL), or lymphoma (e.g., T-cell lymphoma), each characterized by the presence of a mutant allele of IDH1.

### SUMMARY

The present disclosure relates to the glutaric acid cocrystal of Compound 1:

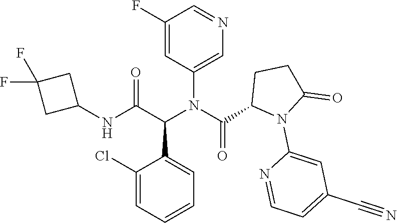 [SMILES: N#Cc1ccnc(N2C(=O)CC[C@H]2C(=O)N(c2cncc(F)c2)[C@H](C(=O)NC2CC(F)(F)C2)c2ccccc2Cl)c1]

Compound 1 is (S)—N—((S)-1-(2-chlorophenyl)-2-((3,3-difluorocyclobutyl)amino)-2-oxoethyl)-1-(4-cyanopyridin-2-yl)-N-(5-fluoropyridin-3-yl)-5-oxopyrrolidine-2-carboxamide, as well as any tautomer or rotamer thereof. Compound 1 is also known as ivosidenib and is marketed under the trade name Tibsovo®.

In one embodiment, the present application relates to a process for the preparation of glutaric acid cocrystal of Compound 1.

In another embodiment, the present application relates to a pharmaceutical composition comprising Compound 1 or the glutaric acid cocrystal of Compound 1 and one or more pharmaceutical excipients.

In another embodiment, the present application relates to a process for the preparation of a pharmaceutical composition comprising Compound 1 or the glutaric acid cocrystal of Compound 1 and one or more pharmaceutical excipients.

In another aspect, the present application relates to a method of treating a cancer characterized by the presence of an IDH1 mutation in a patient in need thereof, comprising administering a therapeutically effective amount of Compound 1 or the glutaric acid cocrystal of Compound 1, or a pharmaceutical composition thereof, to the patient.

In another aspect, the present application relates to Compound 1 or the glutaric acid cocrystal of Compound 1, for use in treating a cancer characterized by the presence of an IDH1 mutation.

### BRIEF DESCRIPTION OF THE DRAWINGS

FIG. 1 depicts the X-ray diffraction spectrum of the glutaric acid cocrystal type A of Compound 1.

FIG. 2 depicts the overlay of the X-ray diffraction spectrum of the glutaric acid cocrystal type A of Compound 1 as recorded (above spectrum) with the theoretical X-ray diffraction spectrum calculated from the single crystal structure (below spectrum).

FIG. 3 depicts the 1H NMR spectrum of the glutaric acid cocrystal type A of Compound 1.

FIG. 4 depicts Differential Scanning Calorimetry (DSC) and Thermogravimetric Analysis (TGA) thermograms of the glutaric acid cocrystal type A of Compound 1.

FIG. 5 depicts the overlay of the X-ray diffraction spectra of the glutaric acid cocrystal type A of Compound 1 stressed 6 weeks at 25° C./60% RH, 25° C./90% RH, 40° C./75% RH and 50° C. with the theoretical X-ray diffraction spectrum of the said cocrystal (calculated form the single crystal structure).

FIG. 6 depicts the overlay of the X-ray diffraction spectra of the glutaric acid cocrystal type A of Compound 1 stressed 12 months at 25° C./60% RH, 25° C./90% RH, 40° C./75% RH and 50° C. with the theoretical X-ray diffraction spectrum of the said cocrystal (calculated form the single crystal structure).

FIG. 7 depicts the sorption isotherm of the cocrystal between 0% RH (relative humidity) to 90% RH at 25° C.

FIG. 8 depicts the dissolution profiles corresponding to the tablets prepared from Form 1, Form T11 or cocrystal of Example 1 or uncoated Tibsovo® tablets recorded as described in Example 6.

FIG. 9 depicts the overlay of the X-ray diffraction spectrum of the glutaric acid cocrystal (either in the lubricated mixture of Examples 3B and 5B or in the tablets of Example 6) stressed 4 weeks at 25° C./60% RH or 40° C./75% RH with the appropriate controls (reference cocrystal, mixtures or tablets before stability test).

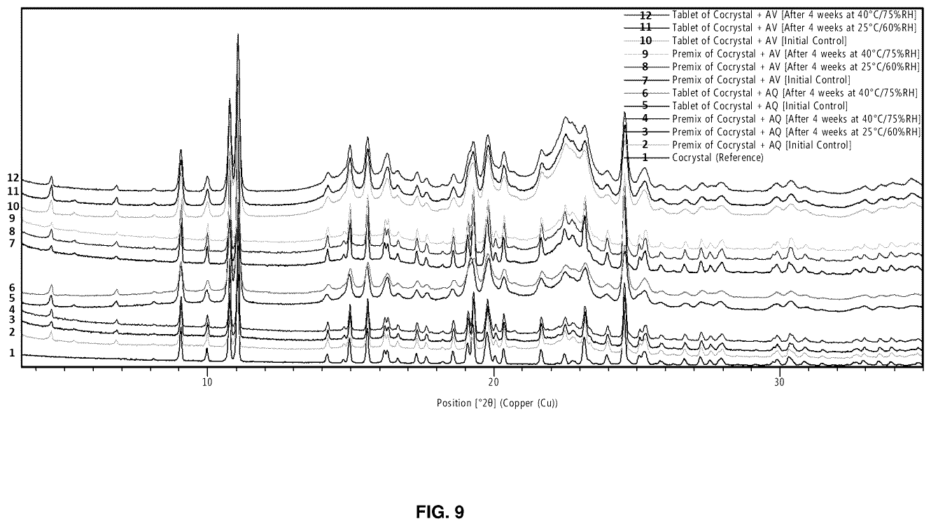

FIG. 10 depicts the 1H NMR spectrum of the glutaric acid cocrystal type A of Compound 1 obtained through the kilolab-scale process of Example 10.

FIG. 11 depicts the overlay of the X-ray diffraction spectrum of the glutaric acid cocrystal type A of Compound 1 obtained through the kilolab-scale process of Example 10 (below spectrum) with the theoretical X-ray diffraction spectrum calculated from the single crystal structure (above spectrum)

### DETAILED DESCRIPTION

The present disclosure relates to glutaric acid cocrystal of Compound 1 pharmaceutical compositions comprising the same, process for the preparation of the same and methods of treatment of cancer, in particular a cancer characterized by the presence of an IDH1 mutation, involving the same.

The present disclosure also relates to glutaric acid cocrystal of Compound 1 for use in treating cancer, in particular a cancer characterized by the presence of an IDH1 mutation.

As used herein, Compound 1 includes the compound having the identified chemical structure, as well as any tautomer or rotamer thereof.

In the specification and claims, each atom of Compound 1 is meant to represent any stable isotope of the specified element. In the Examples, no effort was made to enrich any atom of Compound 1 in a particular isotope, and therefore each atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

In some embodiments, Compound 1 includes each constituent atom at approximately the natural abundance isotopic composition of the specified element.

In one aspect, the disclosure relates to a cocrystal comprising Compound 1

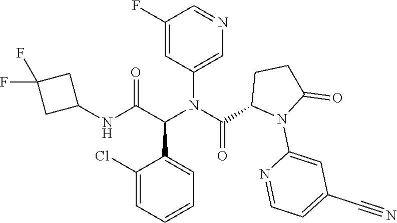 [SMILES: N#Cc1ccnc(N2C(=O)CC[C@H]2C(=O)N(c2cncc(F)c2)[C@H](C(=O)NC2CC(F)(F)C2)c2ccccc2Cl)c1]

and glutaric acid (hereinafter “glutaric acid cocrystal”).

As used herein, the term “cocrystal” refers to a crystalline solid made up of two or more neutral chemical species in a defined stoichiometric ratio that possesses distinct crystallographic and spectroscopic properties when compared to the species individually. A “cocrystal” is distinct from a “salt,” which is made up of charged-balanced charged species. The species making up a cocrystal typically are linked by hydrogen bonding and other non-covalent and non-ionic interactions. Thus, a pharmaceutical cocrystal of a drug typically comprises the drug and one or more coformers. The combinations of drug and coformer(s) that will form cocrystals generally cannot be predicted ab initio, and cocrystal formation typically affects the physicochemical properties of a drug in unpredictable ways.

As used herein, the term “crystalline” refers to a solid material whose constituent particles (e.g., molecules) are arranged spatially in a regular and repeating lattice.

In another aspect, the glutaric acid cocrystal is glutaric acid cocrystal type A.

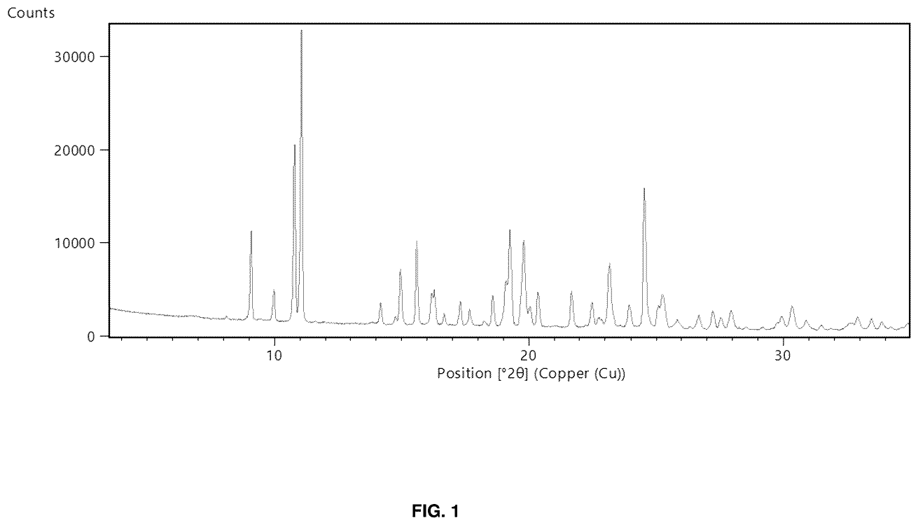

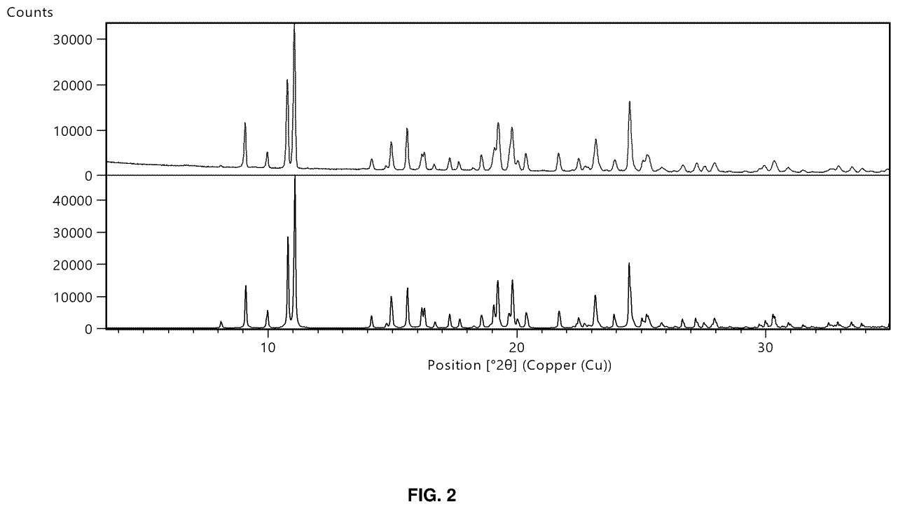

In some embodiments, glutaric acid cocrystal type A is characterized by an X-ray powder diffraction pattern, acquired in transmission mode (sometimes referred to as transmittance mode), comprising one or more peak positions, in degrees 2-theta (±0.2 degrees 2-theta), selected from the peak positions set forth in Table 4 below. In other embodiments, the X-ray powder diffraction pattern comprises at least 1, 2, 3, 4, 5, 6, 7, 8, 9, 10, 11, 12, 13, 14, 15 or all peak positions, in degrees 2-theta (±0.2 degrees 2-theta), selected from the group consisting of 9.1, 10.0, 10.8, 11.1, 15.0, 15.6, 16.2, 16.3, 18.6, 19.1, 19.3, 19.8, 20.4, 21.7, 23.2 and 24.5. In other embodiments, the X-ray powder diffraction pattern comprises at least the following peak positions, in degrees 2-theta (±0.2 degrees 2-theta): 10.8 and 11.1. In other embodiments, the X-ray powder diffraction pattern comprises peak positions, in degrees 2-theta (±0.2 degrees 2-theta), of 10.8 and 11.1, and at least three peak positions selected from the group consisting of 9.1, 10.0, 15.0, 15.6, 16.2, 16.3, 18.6, 19.1, 19.3, 19.8, 20.4, 21.7, 23.2 and 24.5. In other embodiments, the X-ray powder diffraction pattern comprises peak positions, in degrees 2-theta (±0.2 degrees 2-theta), of 9.1, 10.0, 10.8, 11.1, 15.0, 15.6, 16.2, 16.3, 18.6, 19.1, 19.3, 19.8, 20.4, 21.7, 23.2 and 24.5. In other embodiments, the X-ray powder diffraction pattern comprises the peak positions, in degrees 2-theta (±0.2 degrees 2-theta), set forth in Table 4. In other embodiments, the X-ray powder diffraction pattern is similar to the X-ray powder diffraction pattern shown in FIG. 1. In other embodiments, the X-ray powder diffraction pattern is similar to the X-ray powder diffraction pattern shown in FIG. 2.

As used herein, where an X-ray powder diffraction pattern is described as having a specified number of peak positions, “in degrees 2-theta (±0.2 degrees 2-theta),” selected from a specified group of peak positions, the margin of error (±0.2 degrees 2-theta) shall be understood to apply to each peak position within the group.

As used herein, the term “similar,” when referring to two or more X-ray powder diffraction patterns, means that the patterns would be understood by a person of ordinary skill in the art to represent the same crystalline form and that the patterns are the same, except for the types of variations that would be expected by a person of ordinary skill in the art to arise from experimental variations, such as instrumentation used, time of day, humidity, season, pressure, temperature, etc.

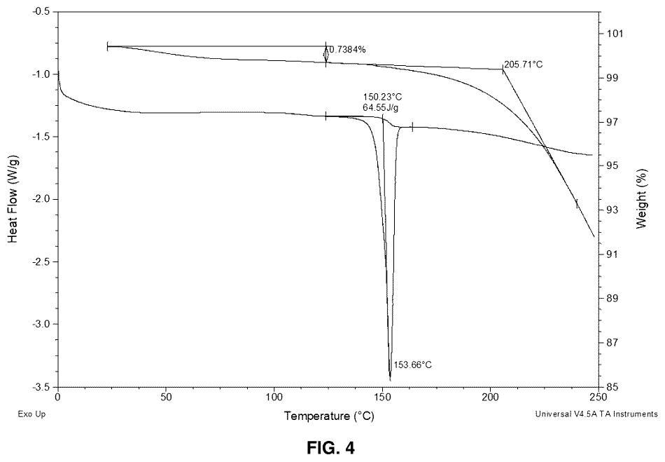

In some embodiments, glutaric acid cocrystal type A is characterized by a differential scanning calorimetry thermogram comprising an endothermic peak having an onset temperature of 150.2° C. (±5.0° C.), as shown in FIG. 4. In other embodiments, glutaric acid cocrystal type A is characterized by a differential scanning calorimetry thermogram comprising an endothermic peak having an onset temperature of 150.2° C. (±2.0° C.), as shown in FIG. 4. In other embodiments, glutaric acid cocrystal type A is characterized by a differential scanning calorimetry thermogram comprising an endothermic peak having a peak temperature of 153.7° C., as shown in FIG. 4.

In some embodiments, the cocrystal comprises Compound 1 and glutaric acid in a molar ratio of 1:1.

In some embodiments, glutaric acid cocrystal type A comprises two molecules of Compound 1 for two glutaric acid molecules per unit cell.

As used herein, the term “unit cell’ refers to the smallest group of particles (e.g., molecules) in a crystalline solid that makes up the repeating pattern of the crystalline solid. In a cocrystal, the term “unit cell” refers to the smallest group of the two or more neutral chemical species that makes up the repeating pattern of the cocrystal.

As discussed in greater detail in the Examples, glutaric acid cocrystal type A was found to have a variety of favorable properties, including high crystallinity, high thermal and physicochemical stability, and a favorable solubility in biorelevant media. Glutaric acid cocrystal type A is also a suitable starting material to prepare tablets containing Compound 1 by direct compression.

In another aspect, the disclosure relates to a process for the preparation of glutaric acid cocrystal of Compound 1 comprising:
dissolving the Compound 1 and glutaric acid in a solvent to afford a solution; and
precipitating the cocrystal.

In some embodiments, the solvent comprises ethyl acetate.

In other embodiments, said precipitating comprises cooling the solution.

In other embodiments, said precipitating comprises adding an antisolvent to the solution, preferably an antisolvent comprising a C₅-C₁₂ alkane or cycloalkane, more preferably an antisolvent comprising heptane.

In other embodiments, said precipitating comprises seeding the solution with crystals of the cocrystal.

In other embodiments, the process further comprises isolating the cocrystal.

### Compositions and Routes of Administration

In another aspect, the disclosure relates to a pharmaceutical composition comprising Compound 1 or glutaric acid cocrystal of Compound 1, as described in any of the embodiments herein, and one or more pharmaceutical excipients.

In another aspect, the disclosure relates to a pharmaceutical composition comprising a therapeutically effective amount of Compound 1 or glutaric acid cocrystal of Compound 1, as described in any of the embodiments herein, and one or more pharmaceutical excipients.

As used herein, the term “therapeutically effective amount,” when referring to an amount of Compound 1 or glutaric acid cocrystal of Compound 1 described herein, refers to an amount that will elicit a biological or medical response in a patient, such as reducing or inhibiting an enzyme or a protein activity, alleviating or ameliorating certain symptoms, curing a disease, lessening the severity of a disease, slowing or delaying the progression of a disease, or preventing a disease. In some embodiments, the term “therapeutically effective amount” refers to the amount of Compound 1 or glutaric acid cocrystal of Compound 1 that, when administered to a patient, is effective to inhibit mutant IDH1. In other embodiments, the term “therapeutically effective amount” refers to the amount of Compound 1 or glutaric acid cocrystal of Compound 1 that, when administered to a patient, is effective to treat a cancer in the patient.

As used herein, the term “pharmaceutical excipient” refers to a carrier, adjuvant, or vehicle that may be administered to a patient together with the Compound 1 or glutaric acid cocrystal of Compound 1, that does not destroy the pharmacological activity of Compound 1, and that is nontoxic when administered in doses sufficient to deliver a therapeutic amount of Compound 1.

Pharmaceutical excipients that may be used in the pharmaceutical compositions described herein include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the Compound 1.

In some cases, the pH of the pharmaceutical composition may be adjusted with pharmaceutically acceptable acids, bases or buffers.

The pharmaceutical compositions described herein may be administered orally, parenterally, by inhalation spray, topically, rectally, nasally, buccally, vaginally or via an implanted reservoir, preferably by oral administration or administration by injection. The pharmaceutical compositions may contain any conventional non-toxic pharmaceutically acceptable excipients.

As used herein, “parenteral” administration includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions may be orally administered in any orally acceptable dosage form including, but not limited to, capsules, tablets, emulsions and aqueous suspensions, dispersions and solutions. In the case of tablets for oral use, excipients which are commonly used include lactose, corn starch, microcrystalline cellulose, croscarmellose sodium, hydroxypropyl cellulose, colloidal silicon dioxide, and sodium lauryl sulfate. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions may also be administered in the form of suppositories for rectal administration. These compositions can be prepared by mixing the Compound 1 or glutaric acid cocrystal of Compound 1 with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions may be administered topically to the skin. The pharmaceutical composition should be formulated with a suitable ointment containing the active components suspended or dissolved in a pharmaceutically acceptable excipient suitable for topical administration, including without limitation mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of one aspect of this invention may also be topically applied to the lower intestinal tract by rectal suppository formulation or in a suitable enema formulation. Transdermal patches are also included in one aspect of this invention.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The amount of active ingredient (e.g., Compound 1) that may be combined with one or more pharmaceutical excipients to produce a single dosage form will vary depending upon the patient treated and the particular mode of administration. A typical preparation will contain from about 5% to about 95% active compound (w/w). Alternatively, such preparations contain from about 20% to about 80% active compound. In some embodiments, the pharmaceutical composition comprises 1-10% w/w of Compound 1 (based on the weight of the free Compound 1, apart from the weight of any coformer, salt former, water of hydration, solvent of solvation, and the like). In some embodiments, the pharmaceutical composition comprises 20-30% w/w of Compound 1 (based on the weight of the free Compound 1, apart from the weight of any coformer, salt former, water of hydration, solvent of solvation, and the like).

The pharmaceutical compositions may further comprise a therapeutically effective amount of an additional therapeutic agent, including without limitation any one of the additional therapeutic agents identified below as being useful in combination therapy.

As used herein, the term “therapeutically effective amount,” when referring to an amount of an additional therapeutic agent, refers to an amount of the agent that will elicit a biological or medical response in a patient, such as reducing or inhibiting an enzyme or a protein activity, alleviating or ameliorating certain symptoms, curing a disease, lessening the severity of a disease, slowing or delaying the progression of a disease, or preventing a disease.

In another aspect, the invention relates to a pharmaceutical composition prepared by a process comprising mixing a therapeutically effective amount Compound 1 or glutaric acid cocrystal of Compound 1, as described in any of the embodiments herein, with one or more pharmaceutical excipients to afford the pharmaceutical composition.

As used here, the term “mixing” means includes any process in which Compound 1 or the glutaric acid cocrystal of Compound 1 is contacted with one or more pharmaceutical excipients to afford a pharmaceutical composition, regardless of whether the pharmaceutical composition so obtained contains Compound 1 or the glutaric acid cocrystal of Compound 1. Thus, the term “mixing” includes processes in which Compound 1 or glutaric acid cocrystal of Compound 1 remains in the same solid form, as well as processes in which Compound 1 or glutaric acid cocrystal of Compound 1 is dissolved and/or converted to a different solid form. Examples of “mixing” processes including wet or dry blending, wet or dry granulation, suspension of Compound 1 or the glutaric acid cocrystal of Compound 1 in the pharmaceutical excipient, and the like.

### Uses of Compound 1 or Glutaric Acid Cocrystal of Compound 1 and Pharmaceutical Compositions Thereof

The inhibitory activities of Compound 1 provided herein against IDH1 mutants (e.g., IDH1R132H or IDH1R132C) can be tested by methods described in Example A of PCT Publication No. WO 2013/107291 or analogous methods.

In another aspect, the invention relates to a method of treating a cancer characterized by the presence of an IDH1 mutation in a patient in need thereof, comprising administering a therapeutically effective amount of Compound 1 or glutaric acid cocrystal of Compound 1, or a pharmaceutical composition thereof, as described in any of the embodiments herein, to the patient.

In another aspect, the invention relates to the use of Compound 1 or a glutaric acid cocrystal of Compound 1, or a pharmaceutical composition thereof, as described in any of the embodiments herein, for the manufacture of a medicament for use in treating a cancer characterized by the presence of an IDH1 mutation in a patient in need thereof.

In another aspect, the invention relates to Compound 1 or glutaric acid cocrystal of Compound 1, or a pharmaceutical composition thereof, as described in any of the embodiments herein, for use in treating a cancer characterized by the presence of an IDH1 mutation in a patient in need thereof.

As used herein, the terms “treat” and “treating,” when referring to a cancer, mean having a therapeutic effect on, alleviating or ameliorating one or more symptoms of, altering the progression of, eradicating, reducing the size of, slowing or inhibiting the growth or progression of, delaying or minimizing one or more symptoms associated with, reducing the malignancy of, or inducing stasis of the cancer. When referring to a disease other than a cancer, the terms “treat” and “treating” mean having a therapeutic effect on, alleviating or ameliorating one or more symptoms of, altering the progression of, eradicating, or delaying or minimizing one or more symptoms associated with the disease.

As used herein, the term “patient” refers to a mammal, including mice, rats, dogs and humans, which is afflicted with a cancer characterized by the presence of an IDH1 mutation. In some embodiments, the patient is a human. In some embodiments, the patient is a human adult (i.e., a human at least 18 years of age). In some embodiments, the patient is a human child (i.e., a human under 18 years of age).

In some embodiments, the cancer is characterized by the presence of an IDH1 mutation. In other embodiments, the IDH1 mutation is an R132X mutation. In other embodiments, the IDH1 mutation is an R132H or R132C mutation. In other embodiments, the IDH1 mutation is an R132H, R132C, R132L, R132V, R132S, or R132G mutation. In other embodiments, the IDH1 mutation is an R132H mutation. In other embodiments, the IDH1 mutation is an R132C mutation. In other embodiments, the IDH1 mutation results in accumulation of R(−)-2-hydroxyglutarate in the patient. In other embodiments, the IDH1 mutation results in a new ability of IDH1 to catalyze the NADPH-dependent reduction of α-ketoglutarate to R(−)-2-hydroxyglutarate. Thus, in some embodiments, treating a cancer characterized by an IDH1 mutation comprises inhibiting mutant IDH1 activity.

In some embodiments, the cancer is a tumor wherein at least 30, 40, 50, 60, 70, 80 or 90% of the tumor cells carry an IDH1 mutation, and in particular an IDH1 R132H or R132C mutation, at the time of diagnosis or treatment.

Without being bound by theory, applicants believe that mutant alleles of IDH1 wherein the IDH1 mutation results in a new ability of the enzyme to catalyze the NADPH-dependent reduction of α-ketoglutarate to R(−)-2-hydroxyglutarate, and in particular R132H and R132C mutations of IDH1, characterize a subset of all types of cancers, without regard to their cellular nature or location in the body. Thus, the compounds and methods of this invention are useful to treat any type of cancer that is characterized by the presence of a mutant allele of IDH1 imparting such activity, and in particular IDH1 R132H and R132C mutations.

As shown in Table 1, IDH1 R132X mutations are known to occur in a variety of cancers.

TABLE 1
Cancers Associated with IDH1 R132X Mutations

| | IDH1 R132X | |
|---|---|---|
| Cancer Type | Mutation | Tumor Type |
| brain tumors | R132H | primary tumor |
| | R132C | primary tumor |
| | R132S | primary tumor |
| | R132G | primary tumor |
| | R132L | primary tumor |
| | R132V | primary tumor |
| fibrosarcoma | R132C | HT1080 fibrosarcoma cell line |
| Acute Myeloid Leukemia | R132H | primary tumor |
| (AML) | R132G | primary tumor |
| | R132C | primary tumor |
| Prostate cancer | R132H | primary tumor |
| | R132C | primary tumor |
| Acute lymphoblastic | R132C | primary tumor |
| leukemia (ALL) | | |
| paragangliomas | R132C | primary tumor |

Accordingly, in some embodiments, the cancer is a cancer selected from the cancer types listed in Table 1, and the IDH1 mutation is one or more of the IDH1 R132X mutations listed in Table 1 for that particular cancer type.

IDH1 R132H mutations have been identified in glioma, acute myelogenous leukemia, sarcoma, melanoma, non-small cell lung cancer, cholangiocarcinomas, chondrosarcoma, myclodysplastic syndromes (MDS), mycloproliferative neoplasm (MPN), colon cancer, and angio-immunoblastic non-Hodgkin's lymphoma (NHL). Accordingly, in some embodiments, the cancer is selected from glioma, acute myelogenous leukemia, sarcoma, melanoma, non-small cell lung cancer (NSCLC), cholangiocarcinomas, chondrosarcoma, myelodysplastic syndromes (MDS), myeloproliferative neoplasm (MPN), colon cancer, or angio-immunoblastic non-Hodgkin's lymphoma (NHL).

A cancer can be analyzed by sequencing cell samples to determine the presence and specific nature of any mutation(s) characterizing the cancer.

In some embodiments, Compound 1 or glutaric acid cocrystal of Compound 1 are useful to treat an advanced hematologic malignancy, e.g., an advanced hematologic malignancy characterized by the presence of a mutant allele of IDH1. In some embodiments, the advanced hematologic malignancy is characterized by a mutant allele of IDH1, wherein the IDH1 mutation results in a new ability of the enzyme to catalyze the NAPH-dependent reduction of α-ketoglutarate to R(−)-2-hydroxyglutarate (2HG) in a patient. In one embodiment, the mutant IDH1 has an R132X mutation. In one embodiment, the R132X mutation is selected from R132H, R132C, R132L, R132V, R132S and R132G. In another aspect, the R132X mutation is R132H or R132C. In one embodiment, the R132X mutation is R132H.

In some embodiments, the advanced hematologic malignancy is characterized by a co-mutation, e.g., a co-mutation selected from NPM1, FLT3, TET2, CEBPA, DNMT3A, and MLL.

In some embodiments, the disorder is selected from acute myelogenous leukemia (AML), myelodysplastic syndrome (MDS), myeloproliferative neoplasms (MPN), chronic myelomonocytic leukemia (CMML), B-acute lymphoblastic leukemias (B-ALL), and lymphoma (e.g., T-cell lymphoma), wherein each is characterized by the presence of a mutant allele of IDH1. In some embodiments, the disorder is selected from advanced IDH1 mutation-positive relapsed and/or refractory AML (R/R AML), untreated AML, and MDS.

In one embodiment, the efficacy of treatment of advanced solid tumors, such as glioma, intrahepatic cholangiocarcinomas (IHCC), chondrosarcoma, prostate cancer, colon cancer, melanoma, or non-small cell lung cancer (NSCLC), each characterized by the presence of a mutant allele of IDH1 is monitored by measuring the levels of 2HG in the subject. Typically levels of 2HG are measured prior to treatment, wherein an elevated level is indicated for the use of Compound 1 or the glutaric acid cocrystal of Compound 1, to treat the advanced solid tumors, such as glioma, intrahepatic cholangiocarcinomas (IHCC), chondrosarcoma, prostate cancer, colon cancer, melanoma, or non-small cell lung cancer (NSCLC), each characterized by the presence of a mutant allele of IDH1. Once the elevated levels are established, the level of 2HG is determined during the course of and/or following termination of treatment to establish efficacy. In certain embodiments, the level of 2HG is only determined during the course of and/or following termination of treatment. A reduction of 2HG levels during the course of treatment and following treatment is indicative of efficacy. Similarly, a determination that 2HG levels are not elevated during the course of or following treatment is also indicative of efficacy. Typically, these 2HG measurements will be utilized together with other well-known determinations of efficacy of cancer treatment, such as reduction in number and size of tumors and/or other cancer-associated lesions, evaluation of bone marrow biopsies and/or aspirates, complete blood counts and examination of peripheral blood films, improvement in the general health of the subject, and alterations in other biomarkers that are associated with cancer treatment efficacy.

2HG can be detected in a sample by the methods of PCT Publication No. WO2011/050210 or by analogous methods.

In some embodiments the cancer is refractory or relapsed. In other embodiments the cancer is newly diagnosed or previously untreated.

In one aspect of this embodiment, the efficacy of cancer treatment is monitored by measuring the levels of 2HG as described herein.

In some embodiments, the efficacy of cancer treatment is monitored by measuring the levels of 2HG in the patient. Typically, levels of 2HG are measured prior to treatment, wherein an elevated level is indicated for the use of Compound 1 or the glutaric acid cocrystal of Compound 1, or pharmaceutical composition thereof, as described in any of the embodiments herein, to treat the cancer. Once the elevated levels are established, the level of 2HG is determined during the course of and/or following termination of treatment to establish efficacy. In certain embodiments, the level of 2HG is only determined during the course of and/or following termination of treatment. A reduction of 2HG levels during the course of treatment and following treatment is indicative of efficacy. Similarly, a determination that 2HG levels are not elevated during the course of or following treatment is also indicative of efficacy. Typically, 2HG measurements will be utilized together with other well-known determinations of efficacy of cancer treatment, such as reduction in number and size of tumors and/or other cancer-associated lesions, improvement in the general health of the patient, and alterations in other biomarkers that are associated with cancer treatment efficacy.

2HG can be detected in a sample by LC/MS. The sample is mixed 80:20 with methanol and centrifuged at 3,000 rpm for 20 minutes at 4 degrees Celsius. The resulting supernatant can be collected and stored at −80 degrees Celsius prior to LC-MS/MS to assess 2-hydroxyglutarate levels. A variety of different liquid chromatography (LC) separation methods can be used. Each method can be coupled by negative electrospray ionization (ESI, −3.0 kV) to triple-quadrupole mass spectrometers operating in multiple reaction monitoring (MRM) mode, with MS parameters optimized on infused metabolite standard solutions. Metabolites can be separated by reversed phase chromatography using 10 mM tributyl-amine as an ion pairing agent in the aqueous mobile phase, according to a variant of a previously reported method (Luo et al. J Chromatogr A 1147, 153-64, 2007). One method allows resolution of TCA metabolites: t=0, 50% B; t=5, 95% B; t=7, 95% B; t=8, 0% B, where B refers to an organic mobile phase of 100% methanol. Another method is specific for 2-hydroxyglutarate, running a fast linear gradient from 50%-95% B (buffers as defined above) over 5 minutes. A Synergi Hydro-RP, 100 mm×2 mm, 2.1 μm particle size (Phenomonex) can be used as the column, as described above. Metabolites can be quantified by comparison of peak areas with pure metabolite standards at known concentration. Metabolite flux studies from ¹³C-glutamine can be performed as described, e.g., in Munger et al. Nat Biotechnol 26, 1179-86, 2008.

In some embodiments, 2HG is directly evaluated.

In other embodiments, a derivative of 2HG formed in the process of performing the analytic method is evaluated. By way of example, such a derivative can be a derivative formed in MS analysis. Derivatives can include a salt adduct, e.g., a Na adduct, a hydration variant, or a hydration variant which is also a salt adduct, e.g., a Na adduct, e.g., as formed in MS analysis.

In another embodiment a metabolic derivative of 2HG is evaluated. Examples include species that build up or are elevated, or reduced, as a result of the presence of 2HG, such as glutarate or glutamate that will be correlated to 2HG, e.g., R-2HG.

Exemplary 2HG derivatives include dehydrated derivatives such as the compounds provided below or a salt adduct thereof:

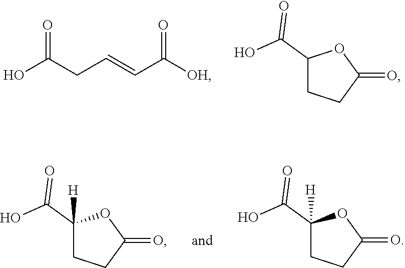 [SMILES: O=C(O)/C=C/CC(=O)O ; O=C1CCC(C(=O)O)O1 ; O=C1CC[C@H](C(=O)O)O1 ; O=C1CC[C@@H](C(=O)O)O1]

In some embodiments, various evaluation steps are performed prior to and/or following treatment of a cancer with Compound 1 or the glutaric acid cocrystal of Compound 1 or pharmaceutical composition thereof. Thus, in some embodiments, the method described herein further comprises an evaluation step prior to and/or after treatment with Compound 1 or the glutaric acid cocrystal of Compound 1 or pharmaceutical composition thereof.

In some embodiments, the evaluation steps comprise evaluating the growth, size, weight, invasiveness, stage and/or other phenotype of the cancer. Thus, in some embodiments, the method described herein further comprises the step of evaluating the growth, size, weight, invasiveness, stage and/or other phenotype of the cancer prior to and/or after treatment with Compound 1 or the glutaric acid cocrystal of Compound 1 or pharmaceutical composition thereof.

In some embodiments, prior to and/or after treatment with Compound 1 or the glutaric acid cocrystal of Compound 1 or pharmaceutical composition thereof, the method further comprises the step of evaluating the IDH1 genotype of the cancer. This may be achieved by ordinary methods in the art, such as DNA sequencing, immuno analysis, and/or evaluation of the presence, distribution or level of 2HG.

In some embodiments, prior to and/or after treatment with Compound 1 or the glutaric acid cocrystal of Compound 1 or pharmaceutical composition thereof, the method further comprises the step of determining the 2HG level in the patient. This may be achieved by spectroscopic analysis, e.g., magnetic resonance-based analysis, e.g., MRI and/or MRS measurement, sample analysis of bodily fluid, such as serum or spinal cord fluid analysis, or by analysis of surgical material, e.g., by mass-spectroscopy.

2HG is known to accumulate in the inherited metabolic disorder 2-hydroxyglutaric aciduria. This disease is caused by deficiency in the enzyme 2-hydroxyglutarate dehydrogenase, which converts 2HG to α-KG (Struys, E. A. et al. Am J Hum Genet 76, 358-60 (2005)). Patients with 2-hydroxyglutarate dehydrogenase deficiencies accumulate 2HG in the brain as assessed by MRI and CSF analysis, develop leukoencephalopathy, and have an increased risk of developing brain tumors (Aghili, M., Zahedi, F. & Rafiee, J Neurooncol 91, 233-6 (2009); Kolker, S., Mayatepek, E. & Hoffmann, G. F. Neuropediatrics 33, 225-31 (2002); Wajner, M., Latini, A., Wyse, A. T. & Dutra-Filho, C. S. J Inherit Metab Dis 27, 427-48 (2004)). Furthermore, elevated brain levels of 2HG result in increased ROS levels (Kolker, S. et al. Eur J Neurosci 16, 21-8 (2002); Latini, A. et al. Eur J Neurosci 17, 2017-22 (2003)), potentially contributing to an increased risk of cancer. The ability of 2HG to act as an NMDA receptor agonist may contribute to this effect (Kolker, S. et al. Eur J Neurosci 16, 21-8 (2002)). 2HG may also be toxic to cells by competitively inhibiting glutamate and/or αKG utilizing enzymes. These include transaminases which allow utilization of glutamate nitrogen for amino and nucleic acid biosynthesis, and αKG-dependent prolyl hydroxylases such as those which regulate HIF1-alpha levels.

Thus, according to another embodiment, one aspect of the invention provides a method of treating 2-hydroxyglutaric aciduria, particularly D-2-hydroxyglutaric aciduria, in a patient by administering to the patient a therapeutically effective amount of Compound 1 or the glutaric acid cocrystal of Compound 1, or a pharmaceutical composition thereof, as described in any one of the embodiments herein.

Also provided are methods of treating a disease selected from Maffucci syndrome and Ollier disease, characterized by the presence of a mutant allele of IDH1 comprising the step of administering to patient in need thereof a therapeutically effective amount of Compound 1 or the glutaric acid cocrystal of Compound 1, or a pharmaceutical composition thereof, as described in any one of the embodiments herein.

Treatment methods described herein can additionally comprise various evaluation steps prior to and/or following treatment with Compound 1 or the glutaric acid cocrystal of Compound 1 or pharmaceutical composition thereof.

In one embodiment, prior to and/or after treatment with Compound 1 or the glutaric acid cocrystal of Compound 1 or pharmaceutical composition thereof, the method further comprises the step of evaluating the growth, size, weight, invasiveness, stage and/or other phenotype of the cancer.

The Compound 1 or the glutaric acid cocrystal of Compound 1, and pharmaceutical compositions thereof, as described in any of the embodiments herein, can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between about 1 mg and about 1000 mg/dose, every 4 to 120 hours, based on the amount of Compound 1. In some embodiments, Compound 1 or glutaric acid cocrystal of Compound 1 or pharmaceutical composition thereof is administered once, twice, or three times a day. In other embodiments, Compound 1 or glutaric acid cocrystal of Compound 1 or pharmaceutical composition thereof is administered once a day. In other embodiments Compound 1 or glutaric acid cocrystal of Compound 1, or pharmaceutical composition thereof is administered twice a day. In other embodiments, Compound 1 or glutaric acid cocrystal of Compound 1, or pharmaceutical composition thereof is administered three times a day. The methods herein contemplate administration of a therapeutically effective amount of Compound 1 or glutaric acid cocrystal of Compound 1, or pharmaceutical composition thereof to achieve the desired or stated effect. Typically, the pharmaceutical compositions of one aspect of this invention will be administered from about 1 to about 6 times per day or alternatively, as a continuous infusion. In some embodiments, Compound 1 or glutaric acid cocrystal of Compound 1, or pharmaceutical composition thereof is administered once a day. In other embodiments, Compound 1 or glutaric acid cocrystal of Compound 1, or pharmaceutical composition thereof is administered twice a day. Such administration can be used as a chronic or acute therapy.

In one embodiment, depending on the disease to be treated and the subject's condition, Compound 1 or the glutaric acid cocrystal of Compound 1 may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. Compound 1 may be formulated alone or together with one or more active agent(s), in suitable dosage unit with pharmaceutically acceptable excipients, carriers, adjuvants and vehicles, appropriate for each route of administration.

All the dosages disclosed herein are based on the amount of Compound 1, that is to say on the amount of Compound 1 free base contained in the administered pharmaceutical composition.

In one embodiment, the amount of Compound 1 administered in the methods provided herein may range, e.g., between about 5 mg/day and about 2,000 mg/day. In one embodiment, the range is between about 10 mg/day and about 2,000 mg/day. In one embodiment, the range is between about 20 mg/day and about 2,000 mg/day. In one embodiment, the range is between about 50 mg/day and about 1,000 mg/day. In one embodiment, the range is between about 100 mg/day and about 1,000 mg/day. In one embodiment, the range is between about 100 mg/day and about 500 mg/day. In one embodiment, the range is between about 150 mg/day and about 500 mg/day. In one embodiment, the range is or between about 150 mg/day and about 250 mg/day. In certain embodiments, particular dosages are, e.g., about 10 mg/day. In one embodiment, the dose is about 20 mg/day. In one embodiment, the dose is about 50 mg/day. In one embodiment, the dose is about 75 mg/day. In one embodiment, the dose is about 100 mg/day. In one embodiment, the dose is about 120 mg/day. In one embodiment, the dose is about 150 mg/day. In one embodiment, the dose is about 200 mg/day. In one embodiment, the dose is about 250 mg/day. In one embodiment, the dose is about 300 mg/day. In one embodiment, the dose is about 350 mg/day. In one embodiment, the dose is about 400 mg/day. In one embodiment, the dose is about 450 mg/day. In one embodiment, the dose is about 500 mg/day. In one embodiment, the dose is about 600 mg/day. In one embodiment, the dose is about 700 mg/day. In one embodiment, the dose is about 800 mg/day. In one embodiment, the dose is about 900 mg/day. In one embodiment, the dose is about 1,000 mg/day. In one embodiment, the dose is about 1,200 mg/day. In one embodiment, the dose is or about 1,500 mg/day. In certain embodiments, particular dosages are, e.g., up to about 10 mg/day. In one embodiment, the particular dose is up to about 20 mg/day. In one embodiment, the particular dose is up to about 50 mg/day. In one embodiment, the particular dose is up to about 75 mg/day. In one embodiment, the particular dose is up to about 100 mg/day. In one embodiment, the particular dose is up to about 120 mg/day. In one embodiment, the particular dose is up to about 150 mg/day. In one embodiment, the particular dose is up to about 200 mg/day. In one embodiment, the particular dose is up to about 250 mg/day. In one embodiment, the particular dose is up to about 300 mg/day. In one embodiment, the particular dose is up to about 350 mg/day. In one embodiment, the particular dose is up to about 400 mg/day. In one embodiment, the particular dose is up to about 450 mg/day. In one embodiment, the particular dose is up to about 500 mg/day. In one embodiment, the particular dose is up to about 600 mg/day. In one embodiment, the particular dose is up to about 700 mg/day. In one embodiment, the particular dose is up to about 800 mg/day. In one embodiment, the particular dose is up to about 900 mg/day. In one embodiment, the particular dose is up to about 1,000 mg/day. In one embodiment, the particular dose is up to about 1,200 mg/day. In one embodiment, the particular dose is up to about 1,500 mg/day.

In one embodiment, the amount of Compound 1 in the pharmaceutical composition or dosage form provided herein may range, e.g., between about 5 mg and about 2,000 mg. In one embodiment, the range is between about 10 mg and about 2,000 mg. In one embodiment, the range is between about 20 mg and about 2,000 mg. In one embodiment, the range is between about 50 mg and about 1,000 mg. In one embodiment, the range is between about 50 mg and about 500 mg. In one embodiment, the range is between about 50 mg and about 250 mg. In one embodiment, the range is between about 100 mg and about 500 mg. In one embodiment, the range is between about 150 mg and about 500 mg. In one embodiment, the range is between about 150 mg and about 250 mg. In certain embodiments, particular amounts are, e.g., about 10 mg. In one embodiment, the particular amount is about 20 mg. In one embodiment, the particular amount is about 50 mg. In one embodiment, the particular amount is about 75 mg. In one embodiment, the particular amount is about 100 mg. In one embodiment, the particular amount is about 120 mg. In one embodiment, the particular amount is about 150 mg. In one embodiment, the particular amount is about 200 mg. In one embodiment, the particular amount is about 250 mg. In one embodiment, the particular amount is about 300 mg. In one embodiment, the particular amount is about 350 mg. In one embodiment, the particular amount is about 400 mg. In one embodiment, the particular amount is about 450 mg. In one embodiment, the particular amount is about 500 mg. In one embodiment, the particular amount is about 600 mg. In one embodiment, the particular amount is about 700 mg. In one embodiment, the particular amount is about 800 mg. In one embodiment, the particular amount is about 900 mg. In one embodiment, the particular amount is about 1,000 mg. In one embodiment, the particular amount is about 1,200 mg. In one embodiment, the particular amount is or about 1,500 mg. In certain embodiments, particular amounts are, e.g., up to about 10 mg. In one embodiment, the particular amount is up to about 20 mg. In one embodiment, the particular amount is up to about 50 mg. In one embodiment, the particular amount is up to about 75 mg. In one embodiment, the particular amount is up to about 100 mg. In one embodiment, the particular amount is up to about 120 mg. In one embodiment, the particular amount is up to about 150 mg. In one embodiment, the particular amount is up to about 200 mg. In one embodiment, the particular amount is up to about 250 mg. In one embodiment, the particular amount is up to about 300 mg. In one embodiment, the particular amount is up to about 350 mg. In one embodiment, the particular amount is up to about 400 mg. In one embodiment, the particular amount is up to about 450 mg. In one embodiment, the particular amount is up to about 500 mg. In one embodiment, the particular amount is up to about 600 mg. In one embodiment, the particular amount is up to about 700 mg. In one embodiment, the particular amount is up to about 800 mg. In one embodiment, the particular amount is up to about 900 mg. In one embodiment, the particular amount is up to about 1,000 mg. In one embodiment, the particular amount is up to about 1,200 mg. In one embodiment, the particular amount is up to about 1,500 mg.

In one embodiment, Compound 1 or the glutaric acid cocrystal of Compound 1 can be delivered as a single dose such as, e.g., a single bolus injection, or oral tablets or pills; or over time such as, e.g., continuous infusion over time or divided bolus doses over time. In one embodiment, Compound 1 or the glutaric acid cocrystal of Compound 1 can be administered repetitively if necessary, for example, until the patient experiences stable disease or regression, or until the patient experiences disease progression or unacceptable toxicity. Stable disease or lack thereof is determined by methods known in the art such as evaluation of patient's symptoms, physical examination, visualization of the tumor that has been imaged using X-ray, CAT, PET, or MRI scan and other commonly accepted evaluation modalities.

In certain embodiments, Compound 1 or the glutaric acid cocrystal of Compound 1 is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, a method provided herein comprises administering Compound 1 or the glutaric acid of Compound 1 in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1. In one embodiment, the median number of cycles administered in a group of patients is about 2. In one embodiment, the median number of cycles administered in a group of patients is about 3. In one embodiment, the median number of cycles administered in a group of patients is about 4. In one embodiment, the median number of cycles administered in a group of patients is about 5. In one embodiment, the median number of cycles administered in a group of patients is about 6. In one embodiment, the median number of cycles administered in a group of patients is about 7. In one embodiment, the median number of cycles administered in a group of patients is about 8. In one embodiment, the median number of cycles administered in a group of patients is about 9. In one embodiment, the median number of cycles administered in a group of patients is about 10. In one embodiment, the median number of cycles administered in a group of patients is about 11. In one embodiment, the median number of cycles administered in a group of patients is about 12. In one embodiment, the median number of cycles administered in a group of patients is about 13. In one embodiment, the median number of cycles administered in a group of patients is about 14. In one embodiment, the median number of cycles administered in a group of patients is about 15. In one embodiment, the median number of cycles administered in a group of patients is about 16. In one embodiment, the median number of cycles administered in a group of patients is about 17. In one embodiment, the median number of cycles administered in a group of patients is about 18. In one embodiment, the median number of cycles administered in a group of patients is about 19. In one embodiment, the median number of cycles administered in a group of patients is about 20. In one embodiment, the median number of cycles administered in a group of patients is about 21. In one embodiment, the median number of cycles administered in a group of patients is about 22. In one embodiment, the median number of cycles administered in a group of patients is about 23. In one embodiment, the median number of cycles administered in a group of patients is about 24. In one embodiment, the median number of cycles administered in a group of patients is about 25. In one embodiment, the median number of cycles administered in a group of patients is about 26. In one embodiment, the median number of cycles administered in a group of patients is about 27. In one embodiment, the median number of cycles administered in a group of patients is about 28. In one embodiment, the median number of cycles administered in a group of patients is about 29. In one embodiment, the median number of cycles administered in a group of patients is about 30. In one embodiment, the median number of cycles administered in a group of patients is greater than about 30 cycles.

In certain embodiments, treatment cycles comprise multiple doses of Compound 1 or glutaric acid cocrystal of Compound 1 administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days).

The amounts of the glutaric acid cocrystal of Compound 1, or pharmaceutical composition thereof, set forth herein are based on the amount Compound 1. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

As used herein, the term “about,” when referring to a dosage, means that the dosage has the specified value±10%. For example, a dosage of “about 100 mg/kg” would include dosages between 90 mg/kg and 110 mg/kg.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of Compound 1, administered as such or as glutaric acid cocrystal, or a pharmaceutical composition thereof, as described in any of the embodiments herein, or combination of one aspect of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Previously Treated Patients

In some embodiments, the patient in need of treatment for a cancer characterized by the presence of an IDH1 mutation was previously administered a cancer therapy. In some embodiments, the patient was previously administered a cancer therapy for the cancer. The previously administered cancer therapy may have been effective or ineffective in treating the cancer or may have been effective for some period of time in treating the cancer.

As used herein, the term “cancer therapy” refers to a cancer therapeutic agent or a cancer treatment. As used herein, the term “cancer therapeutic agent” refers to a therapeutic agent (other than Compound 1 or glutaric acid cocrystal of Compound 1, or the pharmaceutical composition thereof) that is indicated for treating a cancer. Cancer therapeutic agents include, for example, chemotherapy, targeted therapy agents, antibody therapies, immunotherapy agents, hormonal therapy agents, and check point inhibitors. Examples of each of these classes of cancer therapeutic agents are provided below. As used herein, the term “cancer treatment” refers to a treatment that is indicated for treating a cancer. Cancer treatments include, for example, surgery and radiation therapy.

In some embodiments, the cancer therapeutic agent is a chemotherapy agent. Examples of chemotherapy agents used in cancer therapy include, for example, antimetabolites (e.g., folic acid, purine, and pyrimidine derivatives), alkylating agents (e.g., nitrogen mustards, nitrosoureas, platinum, alkyl sulfonates, hydrazines, triazenes, aziridines, spindle poison, cytotoxic agents, topoisomerase inhibitors and others), and hypomethylating agents (e.g., decitabine (5-aza-deoxycytidine), zebularine, isothiocyanates, azacitidine (5-azacytidine), 5-flouro-2′-deoxycytidine, 5,6-dihydro-5-azacytidine and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time. Often, two or more chemotherapy agents are used as combination chemotherapy.

In some embodiments, the cancer therapeutic agent is a differentiation agent. Differentiation agents include retinoids (such as all-trans-retinoic acid (ATRA), 9-cis retinoic acid, 13-cis-retinoic acid (13-cRA) and 4-hydroxy-phenretinamide (4-HPR)); arsenic trioxide; histone deacetylase inhibitors HDACs (such as azacytidine (Vidaza) and butyrates (e.g., sodium phenylbutyrate)); hybrid polar compounds (such as hexamethylene bisacetamide ((HMBA)); vitamin D; and cytokines (such as colony-stimulating factors including G-CSF and GM-CSF, and interferons).

In some embodiments, the cancer therapeutic agent is a targeted therapy agent. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, dasatinib, erlotinib, imatinib, gefitinib, lapatinib, Lestaurtinib, Nilotinib, Semaxanib, Sorafenib, Sunitinib, and Vandetanib, and also cyclin-dependent kinase inhibitors such as Alvocidib and Seliciclib.

Other targeted therapy agents include biguanides such as metformin or phenformin.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®.

In some embodiments, the cancer therapeutic agent is an antibody. Monoclonal antibody therapy is a strategy in which the therapeutic agent is an antibody which specifically binds to a protein on the surface of the cancer cells. Examples include the anti-HER2/neu antibody trastuzumab (HERCEPTIN®) typically used in breast cancer, and the anti-CD20 antibody rituximab and Tositumomab typically used in a variety of B-cell malignancies. Other exemplary antibodies include Cetuximab, Panitumumab, Trastuzumab, Alemtuzumab, Bevacizumab, Edrecolomab, and Gemtuzumab. Exemplary fusion proteins include Aflibercept and Denileukin diftitox.

In some embodiments, the cancer therapeutic agent is an immunotherapy agent. Cancer immunotherapy refers to a diverse set of therapeutic strategies designed to induce the patient's own immune system to fight the tumor. Contemporary methods for generating an immune response against tumors include intravesicular BCG immunotherapy for superficial bladder cancer and use of interferons and other cytokines to induce an immune response in renal cell carcinoma and melanoma patients.

Allogeneic hematopoietic stem cell transplantation can be considered a form of immunotherapy, since the donor's immune cells will often attack the tumor in a graft-versus-tumor effect.

In some embodiments, the cancer therapeutic agent is a hormonal therapy agent. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

In some embodiments, the cancer therapeutic agent is a check point inhibitor. Check point inhibitor therapy is a form of cancer treatment in which manipulation of immune system checkpoints is used restore immune system function against cancer cells. Examples of check point inhibitors include ipilimumab, nivolumab, pembrolizumab, atezolizumab, avelumab, durvalumab, and the like.

Other cancer therapeutic agents include imatinib, gene therapy, peptide and dendritic cell vaccines, synthetic chlorotoxins, radiolabeled drugs and antibodies, Chimeric antigen receptors or CAR-Ts (e.g., Kymriah® (tisagenlecleucel), Yescarta® (axicabtagene ciloleucel)), Gliadel® (carmustine implant), and Avastin® (bevacizumab).

In some embodiments, the cancer treatment is radiation therapy. Radiation therapy involves the use of high-energy radiation (e.g., X-rays, gamma rays, or charged particles) to damage and/or kill cancer cells and to shrink tumors. In the methods of the invention, radiation may be delivered to the brain tumor (e.g., glioma) by a machine positioned outside the body (external-beam radiation therapy), by radioactive material placed in the body near the brain tumor (internal radiation therapy, also called brachytherapy), or by radioactive substances administered systemically (e.g., radioactive iodine) that travel through the bloodstream to the brain tumor. Alternatively, these delivery methods can be used in combination.

In some embodiments, the radiation therapy comprises external radiation therapy (e.g., external-beam radiation therapy including fractionated external-beam radiation therapy, stereotactic radiation such as Cyberknife® or Gamma Knife®, proton therapy, and the like), where the radiation is delivered to the brain tumor (e.g., glioma) by an instrument outside the body. External radiation therapy may be given as a course of several treatments over days or weeks. In one aspect of these embodiments, the radiation is administered in the form of X-rays.

In other embodiments, the radiation therapy comprises internal radiation therapy, where the radiation comes from an implant or a material (liquid, solid, semi-solid or other substance) placed inside the body. In one aspect of these embodiments, the internal radiation therapy is brachytherapy, where a solid radioactive source is placed inside the body near the brain tumor. In another aspect of these embodiments, the internal radiation therapy comprises the systemic administration of a radiation source, typically a radionuclide (radioisotope or unsealed source). The radiation source may be orally administered or may be injected into a vein.

### Combination Therapies

In some embodiments, the methods described herein comprise the additional step of co-administering to a patient in need thereof an additional therapy.

In some embodiments, the medicament for use in treating a cancer characterized by the presence of an IDH1 mutation in a patient in need thereof is for use in combination with the co-administration of an additional therapy.

In another aspect, Compound 1 or the glutaric acid cocrystal of Compound 1, or pharmaceutical composition thereof for use in treating a cancer characterized by the presence of an IDH1 mutation is for use in combination with the co-administration of an additional therapy.

As used herein, the term “additional therapy” includes cancer therapies (including cancer therapeutic agents and cancer treatments), as described above, as well as non-cancer therapies (including non-cancer therapeutic agents and non-cancer treatments) administered to treat symptoms and/or secondary effects of the cancer. In other words, the term “additional therapy” includes additional therapeutic agents (i.e., cancer therapeutic agents and non-cancer therapeutic agents) and additional treatments (i.e., cancer treatments and non-cancer treatments).

In some embodiments, the additional therapy is a cancer therapy (i.e., a cancer therapeutic agent or cancer treatment), as described above.

In some embodiments, the additional therapy is a non-cancer therapy (i.e., a non-cancer therapeutic agent or non-cancer treatment).

In some embodiments, the additional therapy comprises one or more of a DNA-reactive agent, a PARP inhibitor, an anti-emesis agent, an anti-convulsant or anti-epileptic agent, a checkpoint inhibitor, PCV chemotherapy, bevacizumab, and gemcitabine.

In some embodiments, the additional therapy comprises a DNA-reactive agent. As used herein, “DNA-reactive agents” are those agents, such as alkylating agents, cross-linking agents, and DNA intercalating agents, which interact covalently or non-covalently with cellular DNA. For example, DNA-reactive agents include adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, mitozolomide, nedaplatin, oxaliplatin, piposulfan, procarbazine, semustine, streptozocin, temozolomide, thiotepa, treosulfan, diethylnitrosoamine, benzo(a)pyrene, doxorubicin, mitomycin-C, and the like. Many of these DNA-reactive agents are useful in cancer therapy as DNA-reactive chemotherapeutic agents.

In some embodiments, the additional therapy comprises a PARP inhibitor. As used herein, “PARP inhibitor” refers to an inhibitor of the enzyme poly ADP ribose polymerase (PARP). Examples of PARP inhibitors include pamiparib, olaparib, rucaparib, velaparib, iniparib, talazoparib, niraparib, and the like.

In some embodiments, the additional therapy is a checkpoint inhibitor. As used herein, “checkpoint inhibitor” refers to a therapeutic agent that inhibits an immune checkpoint (e.g., CTLA-4, PD-1/PD-L1, and the like) that otherwise would prevent immune system attacks on cancer cells, thereby allowing the immune system to attack the cancer cells. Examples of check point inhibitors include ipilimumab, nivolumab, pembrolizumab, atezolizumab, avelumab, durvalumab, BGB-A317, spartalizumab, and the like.

In some embodiments, the additional therapy is PCV chemotherapy. As used herein, “PCV chemotherapy” refers to a chemotherapy regimen comprising the combined administration of procarbazine, lomustine (which is sold under the trade name CCNU®), and vincristine (which is sold under the trade name Onocovin®). Typically, the vincristine is administered intravenously, while the procarbazine, and lomustine are administered orally. PCV chemotherapy often is administered in cycles, wherein each cycle comprises a single administration of vincristine and lomustine and a 10-day course of treatment with procarbazine.

In some embodiments, the additional therapy is bevacizumab. Bevacizumab, which is sold under the trade name Avastin®, is a recombinant humanized monoclonal antibody.

In some embodiments, the additional therapy is gemcitabine. Gemcitabine, which is sold under the trade name Gemzar®, is a pyrimidine nucleoside analog.

In some embodiments, the additional therapy is a non-cancer therapeutic agent. As used herein, the term “non-cancer therapeutic agent” refers to a therapeutic agent that is used to treat symptoms suffered by patients afflicted with a cancer, and/or undergoing treatment for a cancer, but that is not indicated for treating the cancer itself. Examples of “non-cancer therapeutic agents” include anti-seizure and anti-epileptic agents, anti-emesis agents, anti-diarrheal agents, and the like.

In some embodiments, the additional therapy is an anti-seizure or anti-epileptic agent. As used herein, “anti-seizure or anti-epileptic agent” refers to a drug that is effective for treating or preventing seizures, including epileptic seizures. Examples of anti-seizure and anti-epileptic agents include acetazolamide, barbexaclone, beclamide, brivaracetam, cannabidiol, carbamazepine, clobazam, clonazepam, clorazepate, diazepam, divalproex sodium, eslicarbazepine acetate, ethadione, ethosuximide, ethotoin, etiracetam, felbamate, fosphenytoin, gabapentin, lacosamide, lamotrigine, levetiracetam, lorazepam, mephenytoin, mesuximide, methazolamide, methylphenobarbital, midazolam, nimetazepam, nitrazepam, oxcarbazepine, paraldehyde, paramethadoine, perampanel, piracetam, phenacemide, pheneturide, phenobarbital, phensuximide, phenytoin, potassium bromide, pregabalin, primidone, progabide, pyridoxine, rufinamide, seletracetam, sodium valproate, stiripentol, sultiame, temazepam, tiagabine, topiramate, trimethadione, valnoctamide, valproic acid, valpromide, vigabatrin, zonisamide, and the like.

In some embodiments, the additional therapy is an anti-emesis agent. As used herein, “anti-emesis agent” refers to a drug that is effective to reduce vomiting and nausea symptoms. Examples of anti-emesis agents include 5-HT₃ receptor antagonists (e.g., dolasetron, granisetron, ondansetron, tropisetron, palonosetron, mirtazapine, and the like), dopamine agonists (e.g., domperidone, olanzapine, droperidol, haloperidol, chlorpromazine, prochlorperazine, alizapride, prochlorperazine, metoclopramide, and the like), NK1 receptor antagonists (e.g., aprepitant, casopitant, rolapitant, and the like), antihistamines (e.g., cinnarizine, cyclizine, diphenhydramine, dimenhydrinate, doxylamine, meclizine, promethazine, hydroxyzine, and the like), cannabinoids (e.g, cannabis, dronabinol, synthetic cannabinoids, and the like), benzodiazepines (e.g., midazolam, lorazepam, and the like), anticholinergics (e.g., scopolamine and the like), steroids (e.g., dexamethasone and the like), trimethobenzamide, ginger, propofol, glucose/fructose/phosphoric acid (which is sold under the trade name Emetrol®), peppermint, muscimol, ajwain, bismuth-subsalicylate, and the like.

In some embodiments, the additional therapy is an anti-diarrheal agent. Examples of anti-diarrheal agents include bismuth subgallate, Saccharomyces boulardii lyo, atropine, diphenoxylate, difenoxin, Lactobacillus acidophilus, bismuth subsalicylate, loperamide, Lactobacillus bulgaricus, Lactobacillus rhamnosus gg, attapulgite, crofelemer, simethicone, and the like.

In some embodiments, the additional therapy is a non-cancer treatment. As used herein, the term “non-cancer treatment” refers to a treatment that is used to treat symptoms suffered by patients afflicted with a cancer, and/or undergoing treatment for a cancer, but that is not indicated for treating the cancer itself. Examples of non-cancer treatments include acupuncture, biofeedback, distraction, emotional support and counseling, hypnosis, imagery, relaxation, skin stimulation, and the like.

The term “co-administering” as used herein, means that the additional therapy is administered prior to, concurrently with, consecutively with, or following the administration of the Compound 1 or glutaric acid cocrystal of Compound 1, or pharmaceutical composition thereof as part of a treatment regimen to provide a beneficial effect from the combined action of Compound 1 or glutaric acid cocrystal of Compound 1 (or pharmaceutical composition thereof) and the additional therapy. Where the additional therapy is an additional therapeutic agent, the additional therapeutic agent may be administered together with Compound 1 or glutaric acid cocrystal of Compound 1 as part of a single dosage form (such as a composition of one aspect of this invention comprising a Compound 1 or glutaric acid cocrystal of Compound 1 and the therapeutic agent) or as separate, multiple dosage forms. Alternatively, the therapeutic agent may be administered prior to, consecutively with, or following the administration of Compound 1 or glutaric acid cocrystal of Compound 1. In such combination therapy treatment, both Compound 1 or glutaric acid cocrystal of Compound 1 and the additional therapeutic agent(s) are administered by conventional methods. The administration of a composition of one aspect of this invention, comprising both Compound 1 or glutaric acid cocrystal of Compound 1 and an additional therapeutic agent, to a patient does not preclude the separate administration of that same therapeutic agent, any other additional therapeutic agent or Compound 1 or glutaric acid cocrystal of Compound 1 to said patient at another time during a course of treatment. Where the additional therapy is an additional treatment, the additional treatment may be administered prior to, consecutively with, concurrently with or following the administration of Compound 1 or glutaric acid cocrystal of Compound 1 or pharmaceutical composition thereof.

In some embodiments, when the additional therapy is a cancer therapy, both Compound 1 or glutaric acid cocrystal of Compound 1 and the cancer therapy are administered at dosage levels of between about 1 to 100%, or between about 5 to 95%, of the dosage normally administered in a monotherapy regimen.

### ENUMERATED EMBODIMENTS

In some embodiments, the disclosure relates to:

Embodiment 1. A cocrystal comprising Compound 1:

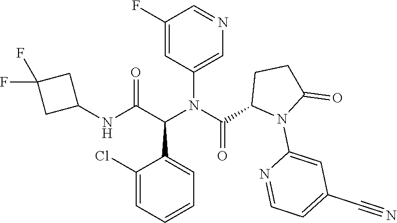 [SMILES: N#Cc1ccnc(N2C(=O)CC[C@H]2C(=O)N(c2cncc(F)c2)[C@H](C(=O)NC2CC(F)(F)C2)c2ccccc2Cl)c1]

and glutaric acid.

Embodiment 2. The cocrystal according to embodiment 1, characterized in that the cocrystal is characterized by an X-ray powder diffraction pattern, acquired in transmission mode, comprising at least 1, 2, 3, 4, 5, 6, 7, 8, 9, 10, 11, 12, 13, 14, 15 or all peak positions, in degrees 2-theta (±0.2 degrees 2-theta), selected from the group consisting of 9.1, 10.0, 10.8, 11.1, 15.0, 15.6, 16.2, 16.3, 18.6, 19.1, 19.3, 19.8, 20.4, 21.7, 23.2 and 24.5.

Embodiment 3. The cocrystal according to embodiment 1, characterized in that the X-ray powder diffraction pattern comprises at least the following peak positions, in degrees 2-theta (±0.2 degrees 2-theta): 10.8 and 11.1.

Embodiment 4. The cocrystal according to embodiment 1, characterized in that the X-ray powder diffraction pattern comprises peak positions, in degrees 2-theta (±0.2 degrees 2-theta), of 10.8 and 11.1, and at least three peak positions selected from the group consisting of 9.1, 10.0, 15.0, 15.6, 16.2, 16.3, 18.6, 19.1, 19.3, 19.8, 20.4, 21.7, 23.2 and 24.5.

Embodiment 5. The cocrystal according to embodiment 1, characterized in that the cocrystal is characterized by an X-ray powder diffraction pattern, acquired in transmission mode, comprising the peak positions, in degrees 2-theta (±0.2 degrees 2-theta), set forth in Table 4.

Embodiment 6. The cocrystal according to any one of embodiments 1 to 5, characterized in that the cocrystal is characterized by a differential scanning calorimetry thermogram comprising an endothermic peak having an onset temperature of 150.2° C. (±5.0° C.).

Embodiment 7. The cocrystal according to any one of embodiments 1 to 6, characterized in that Compound 1 and glutaric acid are present in a molar ratio of 1:1.

Embodiment 8. A pharmaceutical composition comprising a therapeutically effective amount of the cocrystal according to any one of embodiments 1 to 7 and one or more pharmaceutical excipients.

Embodiment 9. The pharmaceutical composition according to embodiment 8, characterized in that the pharmaceutical composition comprises 1-10% w/w of Compound 1.

Embodiment 10. The pharmaceutical composition according to embodiment 8 or 9, characterized in that the pharmaceutical composition is in the form of an orally acceptable dosage form and comprises about 10 mg, about 20 mg, about 50 mg, about 100 mg, about 200 mg, about 250 mg, about 300 mg, or about 500 mg of Compound 1.

Embodiment 11. The pharmaceutical composition according to embodiment 10, characterized in that the pharmaceutical composition comprises about 250 mg of Compound 1.

Embodiment 12. The pharmaceutical composition according to embodiment 8, characterized in that the pharmaceutical composition comprises 20-30% w/w of Compound 1.

Embodiment 13. The pharmaceutical composition according to embodiment 12, characterized in that the pharmaceutical composition is in the form of an orally acceptable dosage form and comprises about 10 mg, about 20 mg, about 50 mg, about 100 mg, about 200 mg, about 250 mg, about 300 mg, or about 500 mg of Compound 1.

Embodiment 14. The pharmaceutical composition according to embodiment 13, characterized in that the pharmaceutical composition comprises about 250 mg of Compound 1.

Embodiment 15. A process for preparation of a pharmaceutical composition according to any one of embodiments 8 to 14 comprising mixing a therapeutically effective amount of the cocrystal of any one according to embodiments 1 to 7 with one or more pharmaceutical excipients to afford the pharmaceutical composition.

Embodiment 16. A process for the preparation of a cocrystal according to any one of embodiments 1 to 7 comprising:
dissolving the Compound 1 and glutaric acid in a solvent to afford a solution; and
precipitating the cocrystal.

Embodiment 17. The process according to embodiment 16, characterized in that the solvent comprises ethyl acetate.

Embodiment 18. The process according to embodiment 16 or 17, characterized in that said precipitating comprises cooling the solution.

Embodiment 19. The process according to any one of embodiments 16 to 18, characterized in that said precipitating comprises adding an antisolvent to the solution.

Embodiment 20. The process according to embodiment 19, characterized in that the antisolvent comprises a C₅-C₁₂ alkane or cycloalkane.

Embodiment 21. The process according to embodiment 20, characterized in that the antisolvent comprises heptane.

Embodiment 22. The process according to any one of embodiments 16 to 21, characterized in that said precipitating comprises seeding the solution with crystals of the cocrystal.

Embodiment 23. The process according to any one of embodiments 16 to 22, further comprising isolating the cocrystal.

Embodiment 24. A process for the preparation of a cocrystal according to any one of embodiments 1 to 7 comprising:
dissolving Compound 1 and glutaric acid in a solvent to afford a solution under stirring,
adding an antisolvent to the solution obtained while stirring,
heating the mixture under stirring,
cooling the mixture under stirring,
filtering the mixture to isolate the cocrystal.

Embodiment 25. The process according to embodiment 24, characterized in that the quantity of glutaric acid is comprised between 1.1 and 1.15 eq. calculated from the molar quantity of Compound 1.

Embodiment 26. The process according to embodiment 24 or 25, characterized in that the solvent comprises ethyl acetate.

Embodiment 27. The process according to embodiment 26, characterized in that Compound 1 and glutaric acid are dissolved in ethyl acetate at 25° C.±2° C.

Embodiment 28. The process according to any one of embodiments 24 to 27, characterized in that the antisolvent comprises a C₅-C₁₂ alkane or cycloalkane.

Embodiment 29. The process according to embodiment 28, characterized in that the antisolvent comprises heptane.

Embodiment 30. The process according to embodiment 29, characterized in that heptane is added step by step.

Embodiment 31. The process according to embodiment 29 or 30, characterized in that the mixture is heated at 70° C.±2° C. after addition of heptane.

Embodiment 32. The process according to any one of embodiments 24 to 31, characterized in that the mixture is cooled down to 10° C.±2° C.

Embodiment 33. The process according to any one of embodiments 24 to 32, characterized in that the stirring is perform at 170 rpm±20 rpm.

Embodiment 34. A cocrystal according to anyone of embodiments 1 to 7 or a pharmaceutical composition according to any one of embodiments 8 to 14 for use in the treatment of a cancer characterized by the presence of an IDH1 mutation.

Embodiment 35. The cocrystal or pharmaceutical composition for use according to embodiment 34, characterized in that the IDH1 mutation is an R132X mutation.

Embodiment 36. The cocrystal or pharmaceutical composition for use according to embodiment 35, characterized in that the IDH1 mutation is an R132H, R132C, R132S, R132L or R132G mutation.

Embodiment 37. The cocrystal or pharmaceutical composition for use according to embodiment 36 characterized in that the IDH1 mutation is an R132H or an R132C mutation.

Embodiment 38. The cocrystal or pharmaceutical composition for use according to any one of embodiments 34 to 37, characterized in that the IDH1 mutation results in accumulation of R(−)-2-hydroxyglutarate in the patient.

Embodiment 39. The cocrystal or pharmaceutical composition for use according to any one of embodiments 34 to 38, characterized in that the cancer is an advanced solid tumor.

Embodiment 40. The cocrystal or pharmaceutical composition for use according to embodiment 39, characterized in that the advanced solid tumor is glioma, intrahepatic cholangiocarcinomas (IHCC), chondrosarcoma, prostate cancer, colon cancer, melanoma, and non-small cell lung cancer (NSCLC).

Embodiment 41. The cocrystal or pharmaceutical composition for use according to any one of embodiments 34 to 38, characterized in that the cancer is an advanced hematologic malignancy.

Embodiment 42. The cocrystal or pharmaceutical composition for use according to embodiment 41, characterized in that the advanced hematologic malignancy is acute myelogenous leukemia (AML), myelodysplastic syndrome (MDS), myeloproliferative neoplasms (MPN), chronic myelomonocytic leukemia (CMML), B-acute lymphoblastic leukemias (B-ALL) and lymphoma.

Embodiment 43. The cocrystal or pharmaceutical composition for use according to any one of embodiments 34 to 42, characterized in that the cancer is refractory or relapsed.

Embodiment 44. The cocrystal or pharmaceutical composition for use according to any one of embodiments 34 to 43, characterized in that the cancer is newly diagnosed or previously untreated.

Embodiment 45. A method of treating a cancer characterized by the presence of an IDH1 mutation in a patient in need thereof, comprising administering a therapeutically effective amount of the cocrystal according to any one of embodiments 1 to 7 or a pharmaceutical composition according to any one of embodiments 8 to 14 to the patient.

Embodiment 46. The method according to embodiment 45, characterized in that the IDH1 mutation is an R132X mutation.

Embodiment 47. The method according to embodiment 46 characterized in that the IDH1 mutation is an R132H, R132C, R132L, R132V, R132S and R132G.

Embodiment 48. The method according to embodiment 47, characterized in that the IDH1 mutation is an R132H or R132C mutation.

Embodiment 49. The method according to any one of embodiments 45 to 48 characterized in that the IDH1 mutation results in accumulation of R(−)-2-hydroxyglutarate in the patient.

Embodiment 50. The method according to any one of embodiments 45 to 49, characterized in that the cancer is an advanced solid tumor.

Embodiment 51. The method according to embodiment 50, characterized in that the advanced solid tumor is glioma, intrahepatic cholangiocarcinomas (IHCC), chondrosarcoma, prostate cancer, colon cancer, melanoma, and non-small cell lung cancer (NSCLC).

Embodiment 52. The method according to any one of embodiments 45 to 49 characterized in that the cancer is an advanced hematologic malignancy.

Embodiment 53. The method according to embodiment 52, characterized in that the advanced hematologic malignancy is acute myelogenous leukemia (AML), myelodysplastic syndrome (MDS), myeloproliferative neoplasms (MPN), chronic myelomonocytic leukemia (CMML), B-acute lymphoblastic leukemias (B-ALL) and lymphoma.

Embodiment 54. The method according to any one of embodiments 45 to 53, characterized in that the cancer is refractory or relapsed.

Embodiment 55. The method according to any one of embodiments 45 to 54 characterized in that the cancer is newly diagnosed or previously untreated.

### EXAMPLES

TABLE 2
Lexicon

| | |
|---|---|
| FaSSIF | Fasted State Simulated Intestinal Fluid |
| SGF | Simulated Gastric Fluid |
| s | singulet |
| d | doublet |
| t | triplet |
| m | multiplet |
| N | newton |
| kN | kilonewton |
| MPa | megapascal |
| rpm | revolution per minute |
| RH | relative humidity |
| SDS | sodium dodecylsulfate |
| AQ | Hypromellose acetate succinate Agoat ® AS-MMP |
| AV | Microcrystalline cellulose Avicel ® PH 302 |
| SD | Standard Deviation |
| TFA | trifluoroacetic acid |
| eq. | equivalent(s) |
| dm/dt | percentage rate of change of mass with time |

Free form 1 of Compound 1 is disclosed and characterized in PCT publication WO2015/138839 and can be prepared according to methods described therein (Example 2).

Free form T11 of Compound is disclosed in PCT publication WO2019/104318 and can be prepared according to methods described therein (Example 12).

### Instrumentation and Methods

Single Crystal X-Ray Diffraction (SCXRD) Analysis A suitable single crystal was selected and mounted in a loop using mineral oil on a XtaLAB Mini (ROW) diffractometer equipped with a MoKα radiation (l=0.71073 Å) at 50 kV and 12 mA. The crystal was kept at 296 K during data collection.

### X-Ray Powder Diffraction (XRPD) Analysis

The XRPD patterns were recorded from 3.5° 2q to 35° 2q using an Empyrean diffractometer from Panalytical operating in the transmission mode with CuKα radiation (l=1.5418 Å) at 45 kV and 40 mA and with a 0.013° 2q step size for 15 minutes (list of examples).

### ¹H NMR Analysis

¹H Liquid NMR spectra were collected on a Bruker 400 MHz NMR Spectrometer. The chemical shifts, in ppm, are given with respect to tetramethylsilane (TMS), using DMSO as internal standard.

### DSC/TGA Analysis

The DSC (Differential Scanning Calorimetry) analyses were recorded from 0° C. to 180° C. or 250° C. (depending on the nature of the crystalline phase) at 10° C./min using a DSC Q2000 from TA instrument for crystalline phase characterization.

The TG (Thermogravimetric) analyses of the cocrystal was recorded from 25° C. to 250° C. at 10° C./min using a TGA Q5000 from TA instrument.

### Example 1. Preparation of Glutaric Acid Cocrystal Type A

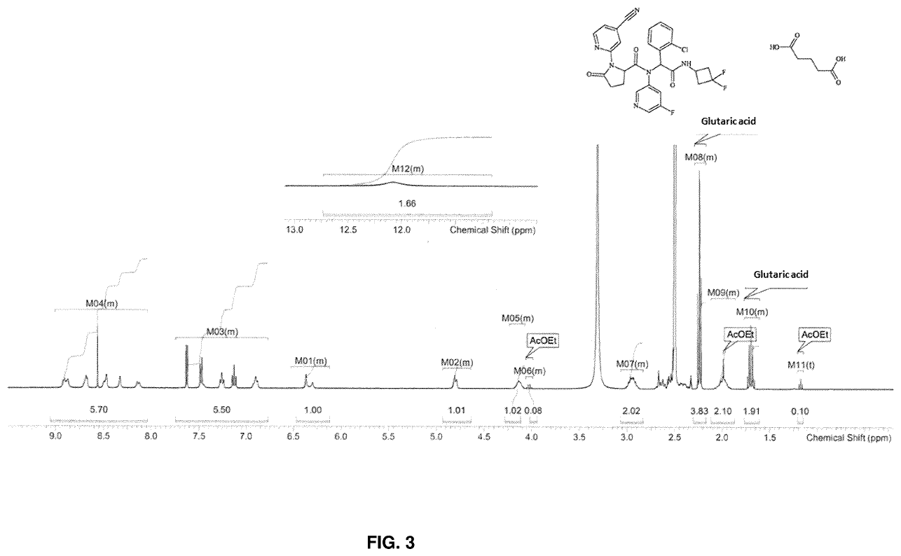

Using a Polar Bear crystallization reactor, in a 500 ml round bottom flask were mixed 6.02 g of Compound 1 (Form 1) with 1.57 g of glutaric acid (1.15 molar equivalent). Under stirring (500 rpm) at room temperature 78 ml of ethyl acetate were added leading to a complete solubilization of the solids. 150 ml of heptane were further added portion wise leading to a precipitate. The mixture was left under stirring for 10 min at room temperature then heated up to 70° C. at 1° C./min. The warm mixture was left 2 h under stirring at 70° C. then cooled down to 20° C. at 0.1° C./min followed by a further stirring at 20° C. for about 11 h. The precipitate was then filtered and dried 24 h at 40° C. under 1 mbar vacuum leading to 5.8 g (79% Yield) of the Compound 1 glutaric acid 1:1 cocrystal in a pure phase (see FIG. 1). Chemical purity as well as stoichiometry were confirmed by complementary ¹H-NMR (see FIG. 3).

¹H NMR (DMSO-d⁶) δ 12.10 (m, 2H), 8.90 (m, 1H), 8.68 (m, 1H), 8.56 (m, 1H), 8.48 (m, 1H), 8.32 (m, 1H), 8.12 (m, 1H), 7.63 (m, 1H), 7.46 (m, 1H), 7.26 (m, 1H), 7.12 (m, 1H), 6.90 (m, 1H), 6.32 (m, 1H), 4.79 (m, 1H), 4.15 (m, 1H), 2.95 (m, 2H), 2.59 (m, 2H), 2.40 (m, 2H), 2.23 (m, 4H), 1.99 (m, 2H), 1.69 (m, 2H).

Single crystals suitable for diffraction analysis were obtained by slow evaporation from an ethyl acetate solution. The crystal structure was solved at room temperature thus confirming the chemical structure of the cocrystal as well as the 1:1 molar stoichiometry between Compound 1 and glutaric acid (see Table 3).

TABLE 3
Resolution of crystal structure of the glutaric
acid cocrystal type A of Compound 1

| Identification code | cocrystal Compound 1 Glutaric acid 1:1 |
|---|---|
| Empirical formula | C₃₃H₃₀ClF₃N₆O₇ |
| Formula weight | 715.08 |
| Temperature/K | 296 |
| Crystal system | monoclinic |
| Space group | P2₁ |
| a/Å | 10.6120(11) |
| b/Å | 15.2730(11) |
| c/Å | 11.9038(13) |
| α/° | 90 |
| β/° | 113.924(13) |
| γ/° | 90 |
| Volume/Å³ | 1763.6(3) |
| Z | 2 |
| ρ_{calc}g/cm³ | 1.347 |
| μ/mm⁻¹ | 0.179 |
| F(000) | 740.0 |
| Crystal size/mm³ | 0.339 × 0.118 × 0.078 |
| Radiation | Mo Kα (λ = 0.71073) |
| 2Θ range for data collection/° | 5.1 to 51.362 |
| Index ranges | −12 ≤ h ≤ 12, −18 ≤ |
| | k ≤ 18, −14 ≤ 1 ≤ 14 |
| Reflections collected | 20025 |
| Independent reflections | 6659 [Rᵢₙₜ = 0.0752, R_{sigma} = 0.0931] |
| Data/restraints/parameters | 6659/1/460 |
| Goodness-of-fit on F² | 0.951 |
| Final R indexes [I >= 2σ (I)] | R₁ = 0.0577, wR₂ = 0.1345 |
| Final R indexes [all data] | R₁ = 0.1463, wR₂ = 0.1734 |
| Largest diff. peak/hole/e Å⁻³ | 0.24/−0.23 |
| Flack parameter | 0.01(8) |

Furthermore, it also confirmed the purity of the batch since the experimental XRPD fully matches the theoretical XRPD calculated form the single crystal structure (see FIG. 2).

The most relevant diffraction peaks of the cocrystal structure as summarized in Table 4.

TABLE 4
XRPD peaks of glutaric acid cocrystal type A of Compound 1

| Pos. [°2θ] | d-spacing [Å] | Rel. Int. [%] | Intensity [cts] |
|---|---|---|---|
| 9.1 | 9.7 | 30.7 | 11567.2 |
| 10.0 | 8.9 | 10.9 | 5109.9 |
| 10.8 | 8.2 | 61.5 | 20999.1 |
| 11.1 | 8.0 | 100.0 | 33550.1 |
| 14.2 | 6.2 | 7.4 | 3679.4 |
| 15.0 | 5.9 | 19.1 | 7368.6 |
| 15.6 | 5.7 | 28.6 | 10413.0 |
| 16.2 | 5.5 | 10.7 | 4719.2 |
| 16.3 | 5.4 | 12.1 | 5094.7 |
| 16.7 | 5.3 | 4.1 | 2526.3 |
| 17.3 | 5.1 | 8.3 | 3838.4 |
| 17.7 | 5.0 | 5.8 | 3021.6 |
| 18.6 | 4.8 | 10.5 | 4483.5 |
| 19.1 | 4.6 | 15.4 | 6087.5 |
| 19.3 | 4.6 | 33.1 | 11663.8 |
| 19.8 | 4.5 | 29.8 | 10580.0 |
| 20.0 | 4.4 | 6.8 | 3253.3 |
| 20.4 | 4.4 | 11.8 | 4834.3 |
| 21.7 | 4.1 | 12.2 | 4927.8 |
| 22.5 | 4.0 | 8.6 | 3741.2 |
| 23.2 | 3.8 | 22.1 | 8009.0 |
| 23.9 | 3.7 | 7.9 | 3451.6 |
| 24.5 | 3.6 | 48.0 | 16222.9 |
| 25.1 | 3.5 | 7.7 | 3370.4 |
| 25.3 | 3.5 | 11.4 | 4538.9 |
| 27.2 | 3.3 | 6.1 | 2750.0 |
| 27.5 | 3.2 | 3.8 | 2033.1 |
| 28.0 | 3.2 | 6.3 | 2802.4 |

The cocrystal exhibits a melting point at Tₒₙₛₑₜ=150.2° C. and T_{Peak}=153.7° C. (see FIG. 4). The cocrystal exhibited a 0.738% weight loss up to 205.7° C. followed by a clear-cut degradation at 206° C. (see FIG. 4).

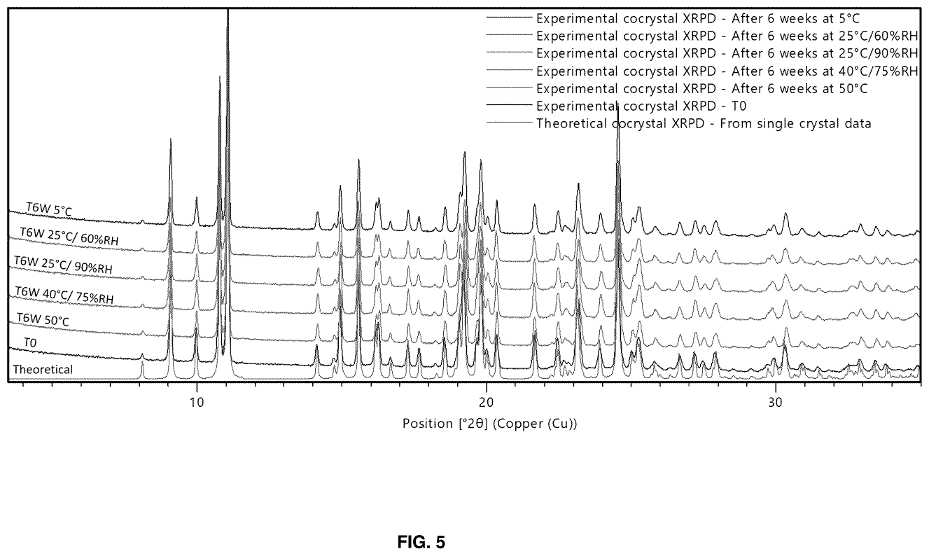

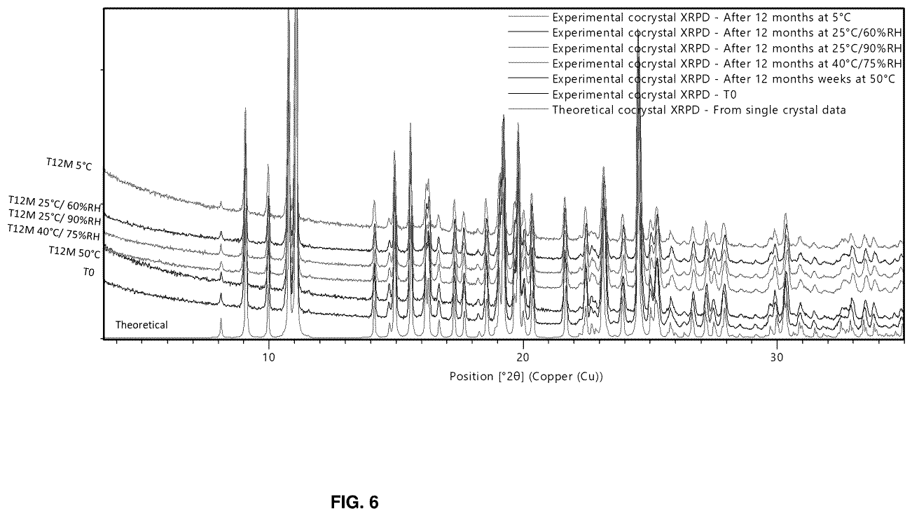

Furthermore, the cocrystal is physicochemically stable after stressing the powder for 6 weeks and 12 months at different temperatures and humidities (6 weeks and 12 months at 25° C./60% RH, 25° C./90% RH, 40° C./75% RH and 50° C., see FIG. 5 and FIG. 6).

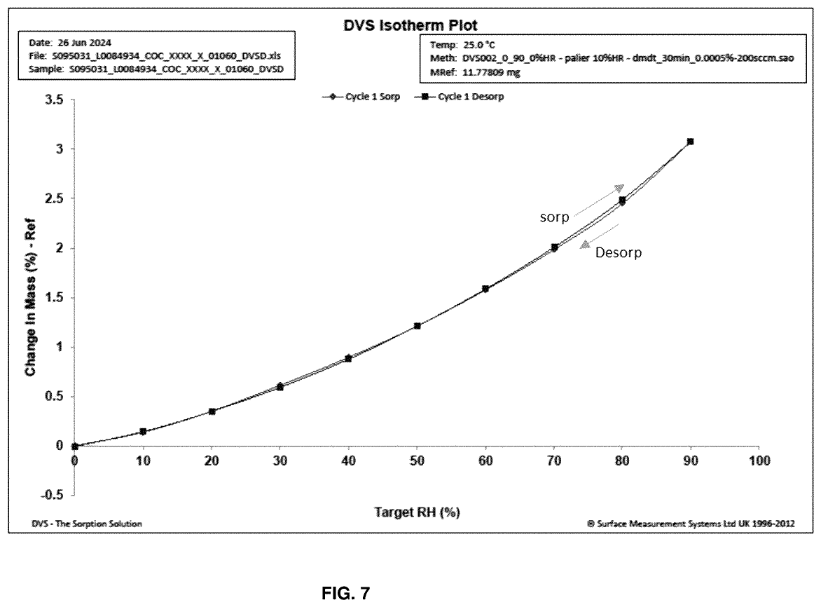

The sorption isotherm of the cocrystal was recorded between 0% RH and 90% RH at 25° C. (see FIG. 7).

The sorption/desorption isotherm of the cocrystal was recorded using the following method:
Equilibrate the sample at 0% RH until the dm/dt was below 0.0005% for 30 consecutive minutes or until maximum 24 hours.
Increase of the relative humidity up to 90% RH with step every 10% RH. Each step was maintained until the dm/dt was below 0.0005% for 30 consecutive minutes or until maximum 24 hours.
Decrease of the relative humidity up to 0% RH with step every 10% RH. Each step was maintained until the dm/dt was below 0.0005% for 30 consecutive minutes or until maximum 24 hours.

The cocrystal is stable in the aforementioned humidity conditions.

The solubility of the glutaric acid cocrystal type A of Compound 1 in biorelevant media was measured both in SGF and FASSIF at 37° C. after 1 h, as shown in Table 5.

TABLE 5
Solubility of the glutaric acid cocrystal of Compound 1

| 37° C., after 1 h | Glutaric acid cocrystal of Compound 1 | |
|---|---|---|
| SGF | 8 | μg/ml |
| FASSIF | 13 | μg/ml |

### Example 2. Preparation of Tablets Containing Compound 1

Tablets were prepared by direct compression using a STYL'ONE EVO compaction simulator from Medelpharm Instruments.

Compound 1, either in Form 1, Form T11 or as a glutaric acid cocrystal type A was lubricated by 0.5% magnesium stearate before compression. 99.5% of Compound 1 and 0.5% of magnesium stearate (e.g., 4.975 g of Compound 1 and 0.025 g of magnesium stearate) were used. Compound 1 was weighed, and magnesium stearate was sifted on a 0.4 mm sieve and added progressively to Compound 1. The mixture was homogenized using a Turbula T2F blender for 5 minutes.

50 mg tablets were prepared using a STYL'ONE EVO compaction simulator applying a compressing force of 2.5 kN or 5.0 kN.

TABLE 6
Tablets characteristics

| Compound 1 | | | | | Glutaric acid | |
|---|---|---|---|---|---|---|
| Sample | Form 1 | | Form T11 | | cocrystal type A | |
| Compressive | 2.5 | 5.0 | 2.5 | 5.0 | 2.5 | 5.0 |
| force applied | | | | | | |
| (kN) | | | | | | |
| Mean | 2.43 | 4.95 | 2.66 | 5.16 | 2.50 | 5.02 |
| compressive | | | | | | |
| force (kN) | | | | | | |
| Tablet hardness | 31.2 | 35.6 | 19.0 | 14.2 | 41.6 | 46.4 |
| (N) | | | | | | |
| Tensile | 1.708 | 1.966 | 1.060 | 1.015 | 2.562 | 2.998 |
| strength (MPa) | | | | | | |

The hardness of the prepared tablets greatly differs depending on the source of Compound 1 used: tablets prepared using glutaric acid cocrystal type A have a better hardness than those prepared using Form 1 and a far better hardness than the ones prepared using Form T11.

As a consequence, tablets prepared from different forms of Compound 1 do not have the same ability to sustain compression: tablets prepared using Form T11 present the lowest ability whereas tablets prepared using glutaric acid cocrystal type A exhibit the highest one among the three sources tested.

Glutaric acid cocrystal type A may therefore be a judicious choice to easily prepare tablets of Compound 1 by direct compression and therefore avoid the preparation of a solid dispersion prior to the manufacturing of tablets.

Commercial tablets of Tibsovo® are prepared from an amorphous solid dispersion using Compound 1 and Aqoat AS-MMP® (HPMC acetate succinate, abbreviated AQ in this section) through a spray drying process.

### Example 3. Preparation of Lubricated Mixtures of Different Forms of Compound 1 with AV and without AQ

### Example 3A. Preparation of the Samples from Form 1 or Form T11 and AV

### 3A1. Preparation of Premix

0.42 g of sodium lauryl sulfate was added to 12.5 g of Compound 1 (either Form 1 or Form T11) in a 220 mL recipient. The mixture was homogenized using a Turbula T2F blender for 5 minutes at 48 rpm. The mixture was then sieved on a 0.8 mm grid which was then rinsed with AV.

### 3A2. Preparation of Mix

25.83 g of AV and 2.5 g of sodium croscarmellose was added in the 220 mL recipient containing the premix obtained following step 3A1. The mixture was homogenized using a Turbula T2F blender for 10 minutes at 48 rpm. The mixture was then sieved on the same 0.8 mm grid.

### 3A3. Preparation of Lubricants Premix

In a 40 mL recipient, 0.21 g of magnesium stearate and 0.21 g of colloidal silica (Aerosil 200®) were introduced with the same volume of the mixture obtained from step 3A2 and mixed by hand. The resulting mixture was then sieved on the same 0.8 mm grid which was rinsed with a little quantity of the mixture obtained from step 3A2.

### 3A4. Lubrication of the Mixture

Lubrication of the mixture obtained from step 3A2 with the lubricants premix of step 3A3 was performed in Turbula T2F blender for 2 minutes at 48 rpm.

### Example 3B. Preparation of the Sample from Glutaric Acid Cocrystal Type a and AV

### 3B1. Preparation of Cocrystal Premix

0.42 g of sodium lauryl sulfate was added to 15.33 g of cocrystal of Example 1 in a 220 mL recipient. The mixture was homogenized using a Turbula T2F blender for 5 minutes at 48 rpm. The mixture was then sieved on a 0.8 mm grid which was then rinsed with AV.

### 3B2. Preparation of Cocrystal Mix

23 g of AV and 2.5 g of sodium croscarmellose was added in the 220 mL recipient containing the premix obtained following step 3B1. The mixture was homogenized using a Turbula T2F blender for 10 minutes at 48 rpm. The mixture was then sieved on the same 0.8 mm grid.

### 3B3. Preparation of the Lubricants Premix

In a 40 mL recipient, 0.21 g of magnesium stearate and 0.21 g of colloidal silica (Aerosil 200®) were introduced with the same volume of the mixture obtained from step 3B2 and mixed by hand. The resulting mixture was then sieved on the same 0.8 mm grid which was rinsed with a little quantity of the mixture obtained from step 3B2.

### 3B4. Lubrication of the Mixture

Lubrication of the mixture obtained from step 3B2 with the lubricants premix of step 3B3 was performed in Turbula T2F blender for 2 minutes at 48 rpm.

### Example 4. Flowability of the Lubricated Mixtures of Examples 3A and 3B

The flowability of the lubricated mixture previously prepared was measured according to the Guidelines of European Pharmacopeia.

Whereas both Form 1 and glutaric acid cocrystal of Compound 1 exhibit a suitable flowability, Form T11 exhibits a poor flowability (refer to Table 7).

TABLE 7
Flowability of the lubricated mixtures of Examples 3A and 3B

| Compound 1 sample | Form 1 | Form T11 | Cocrystal |
|---|---|---|---|
| Flow time (s) | N.S.F. | N.S.F. | 8.3 |
| Carr's Index (%) | 24 | 33 | 24 |
| (flowability) | Adequate | Very poor | Adequate |
| Hausner ratio | 1.32 | 1.49 | 1.32 |
| (cohesion + compressibility) | Adequate | Very poor | Adequate |

N.S.F. = no spontaneous flow detected

### Example 5. Preparation of Lubricated Mixtures of Different Forms of Compound 1 with AQ

### Example 5A. Preparation of the Samples with Form 1 or Form T11 with AQ

### 5A1. Preparation of Premix

0.42 g of sodium lauryl sulfate was added to 12.5 g of Compound 1 (either Form 1 or Form T11) in a 220 mL recipient. The mixture was homogenized using a Turbula T2F blender for 5 minutes at 48 rpm. The mixture was then sieved on a 0.8 mm grid which was then rinsed with AV.

### 5A2. Preparation of Mix

12.50 g of AQ, 13.33 g of AV and 2.5 g of sodium croscarmellose were added in the 220 mL recipient containing the premix obtained following step 5A1. The mixture was homogenized using a Turbula T2F blender for 10 minutes at 48 rpm. The mixture was then sieved on the same 0.8 mm grid.

### 5A3. Preparation of Lubricants Premix

In a 40 mL recipient, 0.21 g of magnesium stearate and 0.21 g of colloidal silica (Aerosil 200®) were introduced with the same volume of the mixture obtained from step 5A2 and mixed by hand. The resulting mixture was then sieved on the same 0.8 mm grid which was rinsed with a little quantity of the mixture obtained from step 5A2.

### 5A4. Lubrication of the Mixture

Lubrication of the mixture obtained from step 5A2 with the lubricants premix of step 5A3 was performed in Turbula T2F blender for 2 minutes at 48 rpm.

### Example 5B. Preparation of the Sample with Glutaric Acid Cocrystal Type a with AQ

### 5B1. Preparation of Cocrystal Premix

0.42 g of sodium lauryl sulfate was added to 15.33 g of cocrystal of Example 1 in a 220 mL recipient. The mixture was homogenized using a Turbula T2F blender for 5 minutes at 48 rpm. The mixture was then sieved on a 0.8 mm grid which was then rinsed with AV.

### 5B2. Preparation of Cocrystal Mix

12.50 g of AQ, 10.50 g of AV and 2.5 g of sodium croscarmellose were added in the 220 mL recipient containing the premix obtained following step 5B1. The mixture was homogenized using a Turbula T2F blender for 10 minutes at 48 rpm. The mixture was then sieved on the same 0.8 mm grid.

### 5B3. Preparation of the Lubricants Premix

In a 40 mL recipient, 0.21 g of magnesium stearate and 0.21 g of colloidal silica (Aerosil 200®) were introduced with the same volume of the mixture obtained from step 5B2 and mixed by hand. The resulting mixture was then sieved on the same 0.8 mm grid which was rinsed with a little quantity of the mixture obtained from step 5B2.

### 5B4. Lubrication of the Mixture

Lubrication of the mixture obtained from step 5B2 with the lubricants premix of step 5B3 was performed in Turbula T2F blender for 2 minutes at 48 rpm.

It was observed that sieving was far more difficult when Form T11 was used instead of Form 1 or glutaric acid cocrystal of Example 1.

### Example 6. Preparation of Tablets by Direct Compression of the Lubricated Mixtures of Examples 3 and 5

The lubricated mixtures previously obtained following Examples 3 and 5 were used to prepare tablets by direct compression using a STYL'ONE EVO compaction simulator from Medelpharm Instruments. The target mass was 833.3 mg, and the target hardness was 265 N.

TABLE 8
Unit formulas of tablets without AQ

| | Form 1 or | Cocrystal of |
|---|---|---|
| Source of Compound 1 | T11 | Example 1 |
| For 1 tablet | In mg | In mg |
| Compound 1 | 249.99 | — |
| Glutaric acid cocrystal (Example 1) | — | 306.63 |
| Microcrystalline cellulose | 516.64 | 460.00 |
| (Avicel PH 302 ®) | | |
| Sodium lauryl sulfate | 8.33 | 8.33 |
| (Kolliphor ® SLS micronized) | | |
| Sodium croscarmellose (AcDiSol ®) | 50.00 | 50.00 |
| Magnesium Stearate MF3 | 4.17 | 4.17 |
| Colloidal silica (Aerosil 200 ®) | 4.17 | 4.17 |
| Total | 833.3 | 833.3 |

TABLE 9
Unit formulas of tablets with AQ

| | Form 1 or | Cocrystal of |
|---|---|---|
| Source of Compound 1 | T11 | Example 1 |
| For 1 tablet | In mg | In mg |
| Compound 1 | 249.99 | — |
| Glutaric acid cocrystal (Example 1) | — | 306.63 |
| HPMC-AS (Aqoat ® AS-MMP) | 249.99 | 249.99 |
| Microcrystalline cellulose | 266.66 | 210.01 |
| (Avicel PH 302 ®) | | |
| Sodium lauryl sulfate | 8.33 | 8.33 |
| (Kolliphor ® SLS micronisé) | | |
| Sodium croscarmellose (AcDiSol ®) | 50.00 | 50.00 |
| Magnesium stearate MF3 | 4.17 | 4.17 |
| Colloidal silica (Aerosil 200 ®) | 4.17 | 4.17 |
| Total | 833.3 | 833.3 |

### Example 7. Dissolution Profile of Tablets of Example 5

900 mL of the dissolution medium (50 mM phosphate buffer with 0.6% SDS at pH 6.8) was dispensed into a dissolution vessel, the paddles were turned on at 50 rpm and the dissolution medium was equilibrated to 37.0±0.5° C. One tablet, previously weighed, was added to the vessel.

Six vessels were prepared following this protocol for each type of tablets (tablet prepared from Form 1, Form T11 or cocrystal of Example 1 or a Tibsovo® tablet prepared as for commercial batches but before coating),

Using a 5 mL disposable syringe fitted with a stainless steel annual and a 10 μm full flow filter, 5 mL of sample solution was withdrawn from a zone midway between the surface of the dissolution medium and the top of the paddle and not less than 1 cm from the vessel walls at the specified time point (sampling window: time point±2%).

Analyses were performed by HPLC using an Inertsil ODS-3 column (5 μm 4.6×50 mm) at 35° C. (the auto sampler was at ambient temperature) using a mixture of acetonitrile/water/TFA (55:45:05 v/v/v) with a 0.8 mL/min flow rate and a 245 nm wavelength. 10 μL were injected and the run time was 6 minutes.

A control sample for HPLC was prepared dissolving 28 mg of Compound 1 in 50 mL of methanol in a 100 mL flask until complete dissolution. Dissolution medium in then added to complete to 100 mL.

TABLE 10
Measured quantities of Compound 1 in solution by HPLC

| | Tibsovo ® | | Tablet prepared | | | | Tablet prepared | | | | Tablet prepared | | | |
|---|---|---|---|---|---|---|---|---|---|---|---|---|---|---|
| | (uncoated tablets) | | from Form 1 | | | | from Form T11 | | | | from Cocrystal | | | |
| | AQ | | AQ | | AV | | AQ | | AV | | AQ | | AV | |
| Diluant | Mean | | Mean | | Mean | | Mean | | Mean | | Mean | | Mean | |
| Time | Value | | Value | | Value | | Value | | Value | | Value | | Value | |
| (min) | (%) | SD. | (%) | SD. | (%) | SD | (%) | SD. | (%) | SD. | (%) | SD. | (%) | SD |
| 0 | 0 | 0 | 0 | 0 | 0 | 0 | 0 | 0 | 0 | 0 | 0 | 0 | 0 | 0 |
| 5 | 40 | 4.39 | 32 | 7.44 | 35 | 4.73 | 9 | 2.42 | 18 | 1.22 | 30 | 3.16 | 26 | 2.62 |
| 10 | 63 | 4.59 | 60 | 4.59 | 49 | 6.16 | 21 | 3.66 | 26 | 1.2 | 51 | 3.83 | 44 | 2.41 |
| 15 | 76 | 4.18 | 68 | 3.54 | 55 | 6.29 | 29 | 3.61 | 30 | 0.98 | 57 | 3.86 | 52 | 3.06 |
| 30 | 92 | 2.18 | 75 | 3.13 | 63 | 5.74 | 38 | 3.1 | 38 | 1.22 | 65 | 3.84 | 61 | 4.38 |
| 45 | 98 | 1.14 | 78 | 2.63 | 66 | 5.41 | 43 | 2.77 | 43 | 1.44 | 68 | 3.34 | 63 | 5.11 |
| 75 | 101 | 0.33 | 95 | 0.46 | 96 | 0.66 | 61 | 2.33 | 62 | 0.89 | 81 | 0.88 | 81 | 0.52 |

The 75 minutes time point is intended to represent an infinite point.

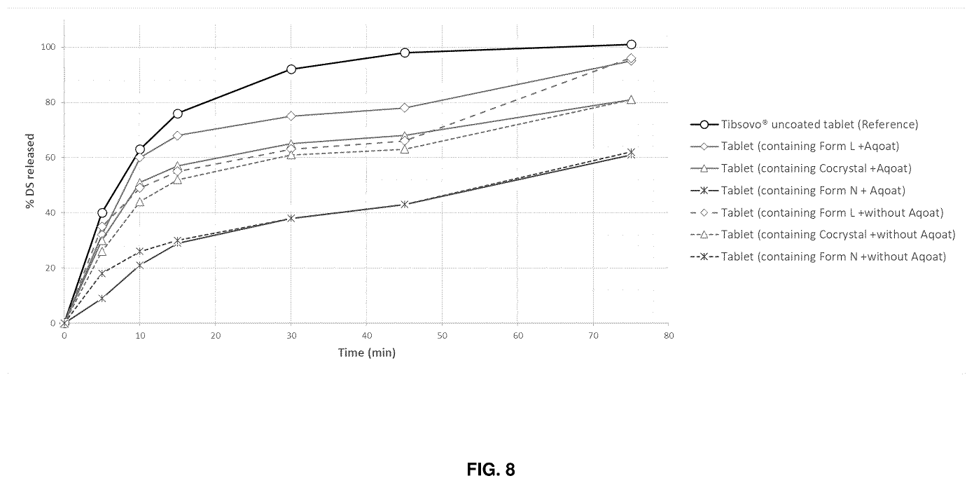

FIG. 8 depicts the corresponding dissolution profiles.

Physical characteristics and properties of glutaric acid cocrystal of Example 1 allow to consider a manufacturing process of tablets with no specific or particular measure. Cocrystal material has comparable behavior with Form 1 regarding handling, flowing properties, cohesion, compressibility, and dissolution profile. In parallel, cocrystal material shows better behavior than Form T11 regarding handling, flowing properties, cohesion and compressibility and dissolution profile.

### Example 8. Comparative Rat PK Study

The objective of this study was to compare the pharmacokinetics (PK) of four oral (PO) dosage forms of Compound 1 in male Wistar rats (4 groups, N=3/group). Lubricated mixtures prepared from Examples 5A (prepared from Form 1 and Form T11) and 5B (prepared from cocrystal of Example 1) and amorphous solid dispersion of Compound 1 obtained through spray drying as in commercial batches were manually and individually filled in capsules (Size 9). The target was of 3 mg of Compound 1 (free base equivalent per capsule) in each capsule.
Dosage Form A: Form 1+AQ (lubricated mixture prepared according to Example 5A)
Dosage Form B: Form T11+AQ (lubricated mixture prepared according to Example 5A)
Dosage Form C: Glutaric acid cocrystal of Example 1+AQ (lubricated mixture prepared according to Example 5B)
Dosage Form D: Amorphous solid dispersion of Compound 1 obtained through spray drying as in commercial batches.

The administered dose was 24 mg/kg, considering that each rat's weight was around 250 g.

Methods: Animals were fasted approximately 6 hours before dosing and fed 3 at 4 hours post-dose. All animals had free access to water. Compound 1 dose were given to rats via oral gavage (2 capsules/one single administration). Blood samples were collected via the saphenous vein into test tubes containing potassium ethylenediaminetetraacetic acid (K2EDTA) from all animals at 0.25, 0.5, 1, 2, 4, 8, 24, 48 and 72 hours post-dose. Blood sample were centrifuged at 2000 g for 5 minutes to obtain plasma samples. Compound 1 concentration in plasma samples was quantified using a liquid chromatography with tandem mass spectrometry (LC-MS/MS) method. The lower limit of quantification was 1.00 ng/mL with a sample volume of 20 μL.

TABLE 11
PK measurements

| | Dosage Form A: | | Dosage Form B: | | Dosage Form C | | Dosage Form D Tibsovo ® | |
|---|---|---|---|---|---|---|---|---|
| | Form 1 + AQ | | Form T11 + AQ | | Cocrystal + AQ | | amorphous solid dispersion | |
| | Mean/Median | | Mean/Median | | Mean/Median | | Mean/Median | |
| PK Parameter | (Tₘₐₓ) | SD | (Tₘₐₓ) | SD | (Tₘₐₓ) | SD | (Tmax) | SD |
| Cₘₐₓ (ng/mL) | 2787 | 701 | 941 | 473 | 2123 | 415 | 3433 | 525 |
| Tₘₐₓ (h) | 8 | — | 8 | — | 8 | — | 8 | — |
| t_{1/2} (h) | 5.8 | 2.9 | 6.8 | 1.4 | 5.2 | 0.8 | 6.5 | 2.1 |

In this experiment, both Form 1 and the glutaric acid cocrystal of Example 1 exhibit a kinetics of absorption which is comparable to the one of the commercial formulation. On the contrary, the absorption profile of Form T11 is very different. The Cmax parameter is drastically diminished, and the absorption occurs over a prolonged time, suggesting a poor absorption process limited by the solubility of the compound. This is confirmed by the observation of an important inter-individual variability (47%) with administration of Form T11 (in contrast, inter-individual variability observed with the administration of Form 1, the cocrystal of Example 1 or the commercial formulation is low, respectively 17.5%, 11% and 10%)

Additionally, Form T11 exhibits an altered bioavailability compared to commercial formulation (43% relative bioavailability) whereas both Form 1 and the cocrystal of Example 1 exhibit acceptable bioavailability compared to the commercial formulation (respectively 78% and 61% relative bioavailability).

All these findings identify the glutaric acid cocrystal of the invention as a promising candidate to further develop an alternative formulation of Compound 1 avoiding the preparation of an intermediate amorphous solid dispersion.

### Example 9. Four Weeks Stability Study

Four weeks stability of samples of lubricated mixtures of Example 3B and 5B and tablets of Example 6 prepared from cocrystal of Example 1 was tested according to the following protocol.

All the samples were stored 4 weeks in controlled temperature and humidity chambers in open vials at 25° C./60% RH or 40° C./75% RH. The XPRD analyses were performed extemporaneously in order to be representative of all the samples at temperature and humidity equilibria.

### X-Ray Powder Diffraction (XRPD) Analysis

Tablets of Cocrystal+AQ/AV or AV (from Example 6) were gently grinded leading a powder suitable for XRPD analysis. Lubricated mixtures from Examples 3B and 5B were analyzed without grinding. The XRPD patterns were recorded from 3.5° 2q to 35° 2q using an Empyrean diffractometer from Panalytical operating in the transmission mode with CuKα radiation (l=1.5418 Å) at 45 kV and 40 mA and with a 0.013° 2q step size for 30 minutes.

The cocrystal of Example 1 is perfectly stable at 25° C./60% RH and 40° C./75% RH in the corresponding lubricated mixtures as well as in the corresponding tablets, either with formulations containing AQ/AV or AV.

### Example 10. Kilolab-Scale Synthesis of Glutaric Acid Cocrystal Type A

In a 10 L reactor equipped with tangential flow stirring (anchor mobile), 277 g (475 mmol, 1 eq.) of Compound 1 was added at ambient temperature then 3243 g (3.59 L) of ethyl acetate under stirring at 170 rpm (corresponding to 110 W/m³). A suspension was obtained after adding 72.22 g (547 mmol, 1.15 eq.) of glutaric acid at ambient temperature.

The suspension was left under stirring for 20 min (170 rpm) at 25° C. to obtain a solution. 4761 g (6.96 L) of heptane were added at 25° C. in 2 hours while maintaining stirring at 170 rpm. During this stage, a suspension was obtained. The suspension was left under stirring at 170 rpm for 10 minutes and then heated up to 70° C. (+1° C./min) and the warm mixture was left under stirring at 70° C. for 2 h.
The suspension was then cooled down to 10° C. (−0.1° C./min), left under stirring at 10° C. for 24 h, then filtered (4 L Stainless steel filter with metallic filter media 105 mm, 20 μm porosity) to afford the glutaric acid cocrystal of type A in an 84% yield.

The cocrystal obtained was analyzed according to the methods below.

### X-Ray Powder Diffraction (XRPD) Analysis

The XRPD patterns were recorded from 4° 2θ to 40° 2θ using an Empyrean diffractometer from Panalytical operating in the transmission mode with CuKα radiation (l=1.5418 Å) at 45 kV and 40 mA and with a 0.013° 2θ step size for 10 minutes.

### ¹H NMR Analysis

1H Liquid NMR spectra were collected on a Bruker 400 MHz NMR Spectrometer. The chemical shifts, in ppm, are given with respect to tetramethylsilane (TMS), using DMSO as internal standard.

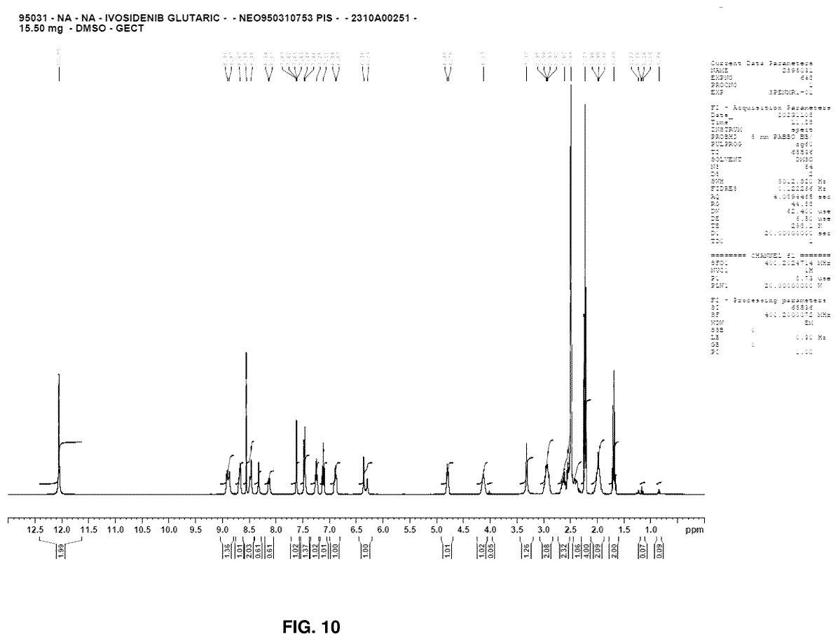

After drying, the cocrystal was obtained with 0.2% for heptane and 0.3% for ethyl acetate as residual solvent (see raw RMN spectrum in FIG. 10).

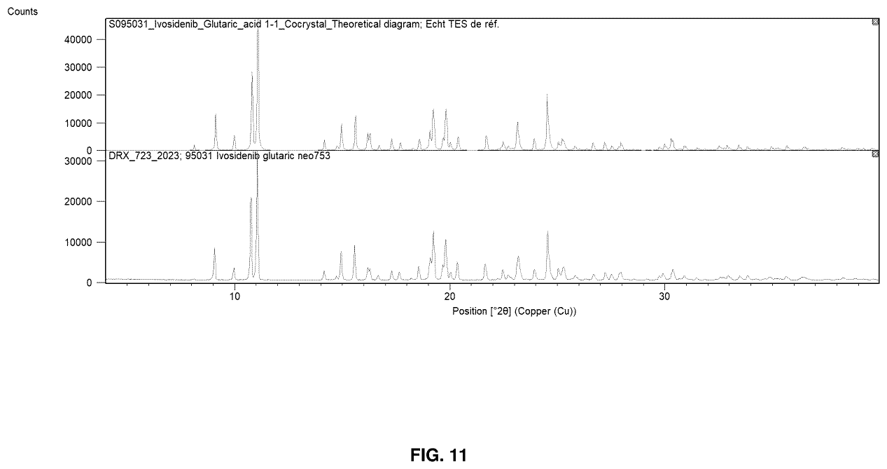

A XRPD diffractogram of the powder obtained was collected and compared to the theoretical diffractogram of the cocrystal (see FIG. 11). It corresponds to the diffractogram of the glutaric acid cocrystal type A.

1. A cocrystal comprising Compound 1 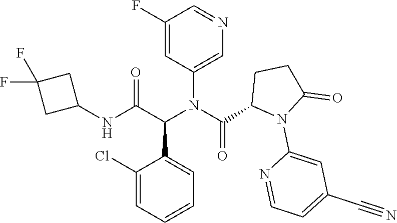 [SMILES: N#Cc1ccnc(N2C(=O)CC[C@H]2C(=O)N(c2cncc(F)c2)[C@H](C(=O)NC2CC(F)(F)C2)c2ccccc2Cl)c1]
and glutaric acid.

2. The cocrystal according to claim 1, wherein the cocrystal is characterized by an X-ray powder diffraction pattern, acquired in transmission mode, comprising at least 1, 2, 3, 4, 5, 6, 7, 8, 9, 10, 11, 12, 13, 14, 15 or all peak positions, in degrees 2-theta (±0.2 degrees 2-theta), selected from the group consisting of 9.1, 10.0, 10.8, 11.1, 15.0, 15.6, 16.2, 16.3, 18.6, 19.1, 19.3, 19.8, 20.4, 21.7, 23.2 and 24.5.

3. The cocrystal according to claim 1, wherein the X-ray powder diffraction pattern comprises at least the following peak positions, in degrees 2-theta (±0.2 degrees 2-theta): 10.8 and 11.1.

4. The cocrystal according to claim 1, wherein the X-ray powder diffraction pattern comprises peak positions, in degrees 2-theta (±0.2 degrees 2-theta), of 10.8 and 11.1, and at least three peak positions selected from the group consisting of 9.1, 10.0, 15.0, 15.6, 16.2, 16.3, 18.6, 19.1, 19.3, 19.8, 20.4, 21.7, 23.2 and 24.5.

5. The cocrystal according to claim 1, wherein the cocrystal is characterized by an X-ray powder diffraction pattern, acquired in transmission mode, comprising the peak positions, in degrees 2-theta (±0.2 degrees 2-theta), set forth in Table 4.

6. The cocrystal according to claim 1, wherein the cocrystal is characterized by a differential scanning calorimetry thermogram comprising an endothermic peak having an onset temperature of 150.2° C. (±5.0° C.).

7. The cocrystal according to claim 1, wherein Compound 1 and glutaric acid are present in a molar ratio of 1:1.

8. A pharmaceutical composition comprising a therapeutically effective amount of the cocrystal according to claim 1 and one or more pharmaceutical excipients.

9. The pharmaceutical composition according to claim 8, wherein the pharmaceutical composition comprises 1-10% w/w of Compound 1.

10. The pharmaceutical composition according to claim 8, wherein the pharmaceutical composition is in the form of an orally acceptable dosage form and comprises about 10 mg, about 20 mg, about 50 mg, about 100_mg, about 200 mg, about 250 mg, about 300 mg, or about 500 mg of Compound 1.

11. The pharmaceutical composition according to claim 10, wherein the pharmaceutical composition comprises about 250 mg of Compound 1.

12. The pharmaceutical composition according to claim 8, wherein the pharmaceutical composition comprises 20-30% w/w of Compound 1.

13. The pharmaceutical composition according to claim 12, wherein the pharmaceutical composition is in the form of an orally acceptable dosage form and comprises about 10 mg, about 20 mg, about 50 mg, about 100_mg, about 200 mg, about 250 mg, about 300 mg, or about 500 mg of Compound 1.

14. The pharmaceutical composition according to claim 13, wherein the pharmaceutical composition comprises about 250 mg of Compound 1.

15-44. (canceled)

45. A method of treating a cancer characterized by the presence of an IDH1 mutation in a patient in need thereof, comprising administering a therapeutically effective amount of the cocrystal according to claim 1 to the patient.

46. The method according to claim 45, wherein the IDH1 mutation is an R132X mutation.

47. The method according to claim 46 wherein the IDH1 mutation is an R132H, R132C, R132L, R132V, R132S and R132G.

48. The method according to claim 47, wherein the IDH1 mutation is an R132H or R132C mutation.

49. The method according to claim 45 wherein the IDH1 mutation results in accumulation of R(−)-2-hydroxyglutarate in the patient.

50. The method according to claim 45, wherein the cancer is an advanced solid tumor.

51. The method according to claim 50, wherein the advanced solid tumor is glioma, intrahepatic cholangiocarcinomas (IHCC), chondrosarcoma, prostate cancer, colon cancer, melanoma, and non-small cell lung cancer (NSCLC).

52. The method according to any claim 45 wherein the cancer is an advanced hematologic malignancy.

53. The method according to claim 52, wherein the advanced hematologic malignancy is acute myelogenous leukemia (AML), myelodysplastic syndrome (MDS), myeloproliferative neoplasms (MPN), chronic myelomonocytic leukemia (CMML), B-acute lymphoblastic leukemias (B-ALL) and lymphoma.

54. The method according to any claim 45, wherein the cancer is refractory or relapsed.

55. The method according to claim 45 wherein the cancer is newly diagnosed or previously untreated.
